(19) United States
(12) Patent Application Publication
(10) Pub. No.: US 2016/0296561 A1
(43) Pub. Date: Oct. 13, 2016

(54) REGULATORY MACROPHAGES AS A CELL-BASED IMMUNOMODULATORY THERAPY IN PULMONARY HYPERTENSION AND RIGHT VENTRICULAR DYSFUNCTION

(71) Applicant: THE BOARD OF TRUSTEES OF THE LELAND STANFORD JUNIOR UNIVERSITY, Stanford, CA (US)

(72) Inventor: Sonja Schrepfer, Stanford, CA (US)

(21) Appl. No.: 15/100,957

(22) PCT Filed: Dec. 1, 2014
(86) PCT No.: PCT/US14/67940

§ 371 Date: Jun. 1, 2016

Related U.S. Application Data
(60) Provisional application No. 61/910,847, filed on Dec. 2, 2013.

(51) Int. Cl. A61K 35/15; C12N 5/0786

(52) U.S. Cl. CPC A61K 35/15; C12N 5/0645; C12N 2501/22; C12N 2501/24

(57) ABSTRACT

Methods and compositions are provided for preventing and treating pulmonary hypertension (PH) and right ventricular (RV) dysfunction. Aspects of the methods include administering to an individual having PH or at risk of developing PH a cell composition that is enriched for regulatory macrophages (Mregs). Also provided are systems, reagents, and kits that find use in practicing the subject methods.

### CROSS-REFERENCE

This application claims the benefit of PCT Application No. PCT/US2014/067940, filed Dec. 1, 2014 and U.S. Provisional Patent Application No. 61/910,847, filed Dec. 2, 2013, which applications are incorporated herein by reference in their entirety.

### FIELD OF THE INVENTION

This disclosure pertains to the prevention and treatment of pulmonary hypertension (PH) and subsequent right ventricular (RV) dysfunction.

### BACKGROUND

Pulmonary arterial hypertension (PH) is a disorder characterized by increased pulmonary vascular resistance due to remodeling and occlusion of the pulmonary arterioles. Chronic PH inevitably results in right ventricular (RV) hypertrophy and dysfunction, even under optimal care management. This eventually leads to RV failure (RVF) from pressure and volume overload resulting in death within 2-3 years. Thus, development of RVF is a strong predictor of poor prognosis in PH. Severity of symptoms and survival are strongly related to the ability of the right heart to overcome the increased afterload. In fact, patients tend to die from severe RV dysfunction rather than from remodeling of the pulmonary vasculature.

Altered immunity and inflammation play a key role in heart failure pathophysiology and are features of PH. This is suggested by infiltration of various inflammatory cells (e.g., macrophages, T and B lymphocytes, etc.), increased cytokine and growth factor (e.g., VEGF and PDGF) expression in remodeled pulmonary vessels, and elevated levels of circulating chemokines and cytokines (e.g. IL-1 and IL-6).

Macrophages belong to a heterogeneous cellular family with various phenotypes sharing a functional adaptability and a high sensibility to the pathological conditions of their environment. They can be involved in both tissue-destructive and tissue-reparative processes. For example, macrophages affect both inflammation and tissue homeostasis. A specific subset of suppressor macrophages, named regulatory macrophages (Mregs), reflects a unique state of macrophage differentiation. Mregs are distinguished from macrophages in other activation states by their particular mode of derivation, robust phenotype, and potent T cell suppressive function. Mregs may represent a particularly suitable cell type for use in clinical tolerance-promoting strategies because autologous Mregs are easily and reliably generated from peripheral blood monocytes and can be safely administered by central venous infusion.

A recent meta-analysis of PH has characterized the field of clinical PH research as “a field in need of new drugs and new study designs.” A better understanding of inflammatory pathways and their role in the pathogenesis of PH may lead to novel therapeutic approaches. There remains an urgent and critical need for methods and composition for treating cardiovascular and pulmovascular diseases, including pulmonary hypertension. The present invention addresses these issues.

### SUMMARY

Methods and compositions are provided for preventing and treating pulmonary hypertension (PH) and subsequent right ventricular (RV) dysfunction. Aspects of the methods include administering to an individual having PH or at risk of developing PH a cell composition that is enriched for regulatory macrophages (Mregs). Also provided are systems, reagents, and kits that find use in practicing the subject methods.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIGS. 1A-1H Injection of SU5416 into athymic RNU rats resulted in severe pulmonary hypertension (PH) and right ventricular (RV) failure (RVF) under normoxic conditions within 28 days. (FIG. 1A) Elastica-van-Gieson staining of lung sections from control and SU5416 injected RNU rats at day 28 post injection. (FIG. 1B) Hemodynamics at 4 weeks in the control group and after SU5416 injection demonstrates a significant increase in right ventricular end-systolic pressure (RVESP), as assessed by PV loops. (FIG. 1C) Confirmation of RV failure in SU5416 injected animals by echocardiography. TAPSE: tricuspid annular plane systolic excursion; RVFAC: right ventricular fractional area change. (FIG. 1D-1E) Echocardiography shows the massive dilated RV at 4 weeks after SU5416-injection. FIG. 1D: control animals. FIG. 1E: animals injected with SU5416. (FIG. 1F) SU5416 injection causes increased pulmonary vascular resistance. Shown is a Pulse Doppler of the pulmonary artery trunk showing a notch in the ejection curve. This highlights the major increase in pulmonary vascular resistance. (FIG. 1G-1H) Chronic pressure overload of the RV results in significant infiltration of CD68+ macrophages into the RV myocardium (4 weeks after SU5416 injection). (FIG. 1G) Confocal immunofluorescence imaging showing the infiltration of macrophages (using an antibody against CD68) in the RV. (FIG. 1H) Quantitation of confocal images (CD68+ macrophages per cell (as determined by DAPI staining)). Note, infiltration was not observed in the LV wall, suggesting that the observations are not related to general SU5416 toxicity. RV: right ventricle; LV: left ventricle.

FIGS. 2A-2H (FIG. 2A) Typical morphology of Mregs generated from spleen and bone marrow (magnification of 200× at day 0 (left) and day 6 (right)). Although Mregs share markers of both resting and M1 macrophages, they can be readily distinguished from these populations. (FIG. 2B) Bright field images showing that Mregs exhibit a clear difference in morphology compared to classically activated macrophages. (FIG. 2C-2H) specific mAb staining (open histogram) and isotype control mAb staining (grayfilled histogram) of various markers.

FIGS. 3A-3D (FIG. 3A) Category “SU5416” represents animals that were injected with SU5416 on day −1, but were not injected with Mregs. Category “control” represents untreated animals. Category “prevention” represents animals that were injected with Mregs on day −1 as described above, and then injected with SU5416 on day 0. (FIG. 3B) remodeling of the pulmonary arteries visualized in histological sections of the pulmonary artery stained with Elastica von Gieson stain. (FIG. 3C) remodeling of the pulmonary arteries: measured by determining medial thickness of the pulmonary artery (FIG. 3D) schematic illustrating medial thickness (MT) as measured in FIG. 3C. *p<0.05 FIG. 4 presents data relating to the effectiveness of the injection of Mregs in preventing RV dysfunction, as measured at day 28 by a number of different parameters related to ventricular structure and function. RVESP: right ventricular end-systolic pressure, as assessed by PV loops. RVFAC: right ventricular fractional area change. TAPSE: tricuspid annular plane systolic excursion. RVEDA: right ventricular end-diastolic area. *p<0.05

FIGS. 5A-5B presents data relating to the effectiveness of the injection of Mregs in preventing RV dysfunction, as measured at day 28 by parameters related to ventricular remodeling. (FIG. 5A) Histological hematoxylin eosin stain of a sample from the right ventrical. (FIG. 5B) RV remodeling assessed by calculating the weight RV ratio (RV/(LV+S)), and by measuring the cardiomyocyte cross-sectional area. *p<0.05

FIGS. 6A-6C presents data relating to the effectiveness of the injection of Mregs in preventing RV dysfunction, as measured at day 28 by parameters related to RV-PA coupling. (FIG. 6A-6B) PV loops for end-systolic pressure-volume analysis of the RV were recorded at 4 weeks after median sternotomy to introduce a conductance catheter into the RV (FIG. 6C) assessment of RV end-systolic elastance (Ees) and PA elastance (Ea) was used to determine the RV-PA coupling ratio (Ees/Ea). *p<0.05

FIGS. 7A-7C presents data relating to the distribution of injected Mregs over time (Single-photon emission computed tomography (SPECT) imaging in vivo). (FIG. 7A-7B) representative images. (FIG. 7C) Quantified results.

FIG. 8 presents data relating to the distribution of injected Mregs over time (Bioluminescence (BLI) imaging of extracted organs).

### DETAILED DESCRIPTION

Methods and compositions are provided for preventing and treating pulmonary hypertension (PH) and right ventricular (RV) dysfunction. Aspects of the methods include administering to an individual having PH or at risk of developing PH a cell composition that is enriched for regulatory macrophages (Mregs). Also provided are systems, reagents, and kits that find use in practicing the subject methods. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods

Aspects of the disclosure include methods for treating an individual having pulmonary hypertension (PH) and for preventing pulmonary hypertension (PH) in an individual at risk for developing pulmonary hypertension (PH). The term “pulmonary hypertension” or “PH” as used herein refers to a type of high blood pressure that affects the pulmonary arteries and the right side of the heart. Pulmonary hypertension is a serious illness that becomes progressively worse and is sometimes fatal. PH is characterized by increased pulmonary vascular resistance due to remodeling and occlusion of the pulmonary arterioles. For example, narrowing, blocking, or destruction of the pulmonary arties and/or lung capillaries makes it difficult for blood to flow through the lungs, raising pressure within the pulmonary arteries. Other factors that can affect the pulmonary arteries and can cause PH to develop include: (i) the walls of the arteries may tighten; (ii) the walls of the arteries may be stiff at birth or become stiff from an overgrowth of cells; and/or (iii) blood clots may form in the arteries. As the pressure within the pulmonary arteries builds, the heart's lower right chamber (right ventricle) must work harder than normal to pump blood through the lungs, which can cause the heart to become strained and weakened, and can eventually cause the heart to fail. Heart failure is the most common cause of death in people who have PH.

As used herein, PH can be defined by a mean pulmonary arterial pressure greater than or equal to 25 mmHg with a capillary wedge pressure less than or equal to 15 mmHg. PH can be divided into five groups based on its causes and treatment options. In all groups, the average pressure in the pulmonary arteries is greater than or equal to 25 mmHg. The pressure in normal pulmonary arteries is 8-20 mmHg at rest. Thus, in some cases, mean pulmonary arterial pressure can be used to determine whether an individual has PH. Other symptoms of PH which can be used to identify an individual as having PH can include any or all of: (i) shortness of breath during routine activity (e.g., climbing two flights of stairs); (ii) tiredness/fatigue; (iii) chest pain or pressure; (iv) a rapid heartbeat; (v) swelling of the ankles, legs, and abdomen; (vi) dizziness; and (vii) decreased appetite. Symptoms of PH can limit an individual's ability to exercise and do other physical activities. As the condition worsens, its symptoms may limit all physical activity. Diagnostic tests for determining whether an individual has PH may include a chest X-ray, a breathing test (e.g. a pulmonary function test), an echocardiogram (also called an “echo”), a measurement of pulmonary arterial pressure, and the like. In some cases, an individual with PH, an individual suspected of having PH, or an individual who is at risk of developing PH can have any or all of the above symptoms, including any combination of the above symptoms (e.g., shortness of breath during routine activity; tiredness/fatigue; chest pain or pressure; a rapid heartbeat; swelling of the ankles, legs, and/or abdomen; dizziness; and decreased appetite).

In some instances, the individual having PH will develop right ventricular dysfunction. By “right ventricular dysfunction” or “RV dysfunction” it is meant a decrease in normal function of the RV. In some instances, RV dysfunction includes severe RV dysfunction, i.e. RV failure. As indicated above, the subject methods may also be employed in preventing RV dysfunction in an individual at risk for developing RV dysfunction.

A number of methods are known in the art for assessing RV function, any of which may be used to determine if, for example, the individual suffers from RV dysfunction. For example, remodeling of the pulmonary artery can be assessed. Any convenient method in the art for assessing remodeling of the pulmonary artery may be employed. For example, the medial thickness of the pulmonary aorta may be measured, where a medial thickening, or an increase thereof, is associated with RV dysfunction. Thus, preventing RV dysfunction in an individual can prevent an increase in medial thickness of the PA compared to the normal medial thickness of the PA for a normal individual (e.g., compared to an individual of comparable age, sex, weight, etc.). Stabilizing RV dysfunction in an individual can prevent an increase in medial thickness of the PA compared to the medial thickness that was present prior to performing the method (e.g., when the medial thickness was already increased compared to normal prior to performing the method). In other words, stabilizing RV dysfunction can prevent further medial thickening of an already thickened PA. Reversing RV dysfunction can reduce the medial thickness of the PA compared to the medial thickness that was present prior to performing the method (e.g., when the medial thickness was already increased compared to normal prior to performing the method). In other words, reversing RV dysfunction can reduce the medial thickness of the PA (e.g., producing a medial thickness that is closer to normal).

As another example, RV dysfunction can be assessed by assessing ventricular structure (e.g., RV structure). Any convenient method in the art for assessing ventricular structure may be employed. For example, right ventricular end-systolic pressure (RVESP), right ventricular fractional area change (RVFAC), tricuspid annular plane systolic excursion (TAPSE), and/or right ventricular end-diastolic area (RVEDA) may be measured, where an increased RVESP and/or increased RVEDA and/or decreased RVFAC and/or decreased TAPSE is (are) associated with RV dysfunction. Thus, preventing RV dysfunction in an individual can prevent an increase in RVESP and/or RVEDA compared to the normal RVESP and/or RVEDA for a normal individual (e.g., compared to an individual of comparable age, sex, weight, etc.). Stabilizing RV dysfunction in an individual can prevent an increase in RVESP and/or RVEDA compared to the RVESP and/or RVEDA that was present prior to performing the method (e.g., when the RVESP and/or RVEDA was already increased compared to normal prior to performing the method). In other words, stabilizing RV dysfunction can prevent a further increase in RVESP and/or RVEDA. Reversing RV dysfunction can reduce the RVESP and/or RVEDA compared to the RVESP and/or RVEDA that was present prior to performing the method (e.g., when the RVESP and/or RVEDA was already increased compared to normal prior to performing the method). In other words, reversing RV dysfunction can reduce the RVESP and/or RVEDA (e.g., producing a RVESP and/or RVEDA that is closer to normal). Likewise, preventing RV dysfunction in an individual can prevent a decrease in RVFAC and/or TAPSE compared to the normal RVFAC and/or TAPSE for a normal individual (e.g., compared to an individual of comparable age, sex, weight, etc.). Stabilizing RV dysfunction in an individual can prevent a decrease in RVFAC and/or TAPSE compared to the RVFAC and/or TAPSE that was present prior to performing the method (e.g., when the RVFAC and/or TAPSE was already decreased compared to normal prior to performing the method). In other words, stabilizing RV dysfunction can prevent a further decrease in RVFAC and/or TAPSE. Reversing RV dysfunction can increase the RVFAC and/or TAPSE compared to the RVFAC and/or TAPSE that was present prior to performing the method (e.g., when the RVFAC and/or TAPSE was already decreased compared to normal prior to performing the method). In other words, reversing RV dysfunction can increase RVFAC and/or TAPSE (e.g., producing a RVFAC and/or TAPSE that is closer to normal).

As another example, RV dysfunction can be assessed by assessing ventricular remodeling (e.g., RV remodeling). Any convenient method in the art for assaying ventricular remodeling may be employed. For example, the weight RV ratio (RV/(LV+S)) may be measured, where an increase in weight RV ratio and/or cardiomyocyte area (e.g., cross-sectional area) is associated with RV dysfunction. Thus, preventing RV dysfunction in an individual can prevent an increase in weight RV ratio and/or cardiomyocyte area compared to the normal weight RV ratio and/or cardiomyocyte area for a normal individual (e.g., compared to an individual of comparable age, sex, weight, etc.). Stabilizing RV dysfunction in an individual can prevent an increase in weight RV ratio and/or cardiomyocyte area compared to the weight RV ratio and/or cardiomyocyte area that was present prior to performing the method (e.g., when the weight RV ratio and/or cardiomyocyte area was already increased compared to normal prior to performing the method). In other words, stabilizing RV dysfunction can prevent a further increase in weight RV ratio and/or cardiomyocyte area. Reversing RV dysfunction can reduce the weight RV ratio and/or cardiomyocyte area compared to the weight RV ratio and/or cardiomyocyte area that was present prior to performing the method (e.g., when the weight RV ratio and/or cardiomyocyte area was already increased compared to normal prior to performing the method). In other words, reversing RV dysfunction can reduce the weight RV ratio and/or cardiomyocyte area (e.g., producing a weight RV ratio and/or cardiomyocyte area that is closer to normal).

As another example, RV dysfunction can be assessed by determining RV-PA coupling. RV-PA coupling can be determined by measuring, for example, RV end-systolic elastance (Ees) and PA elastance (Ea), and calculating the RV-PA coupling ratio (Ees/Ea). A decrease in RV-PA coupling ratio is associated with RV dysfunction. Thus, preventing RV dysfunction in an individual can prevent a decrease in RV-PA coupling ratio compared to the normal RV-PA coupling ratio for a normal individual (e.g., compared to an individual of comparable age, sex, weight, etc.). Stabilizing RV dysfunction in an individual can prevent a decrease in RV-PA coupling ratio compared to the RV-PA coupling ratio that was present prior to performing the method (e.g., when the RV-PA coupling ratio was already decreased compared to normal prior to performing the method). In other words, stabilizing RV dysfunction can prevent a further decrease in RV-PA coupling ratio. Reversing RV dysfunction can increase the RV-PA coupling ratio compared to the RV-PA coupling ratio that was present prior to performing the method (e.g., when the RV-PA coupling ratio was already decreased compared to normal prior to performing the method). In other words, reversing RV dysfunction can increase RV-PA coupling ratio (e.g., producing a RV-PA coupling ratio that is closer to normal).

Additional methods and parameters for assessing RV function and assessing whether an individual has RV dysfunction (or has reversed and/or stabilized RV dysfunction following treatment by the subject methods and/or using the subject compositions) can be found, for example, in the scientific literature: Voelkel et al., Circulation. 2006 Oct. 24; 114(17):1883-91: “Right ventricular function and failure: report of a National Heart, Lung, and Blood Institute working group on cellular and molecular mechanisms of right heart failure.”

Additional information about how to determine whether or not an individual has PH, is suspected of having PH, or is at risk for developing PH can be found, for example, in US patent applications 20110171193, 20120010095, 20120295797, and 20050196868; and in the scientific literature, for example, Galiè et al., Eur Heart J. 2009 October; 30(20):2493-537: “Guidelines for the diagnosis and treatment of pulmonary hypertension”; all of which are hereby incorporated by reference in their entirety.

A number of diseases or conditions, such as heart and lung diseases or blood clots, can cause PH. For example, causes of PH can include: congestive heart failure; birth defects in the heart; chronic pulmonary thromboembolism (blood clots in the pulmonary arteries); acquired immunodeficiency syndrome (AIDS); cirrhosis of the liver; lupus; pulmonary fibrosis; collagen vascular disease; portal hypertension; thyroid disorders; glycogen storage disease; Gaucher's disease; hereditary hemorrhagic telangiectasia; hemoglobinopathies; chronic myeloproliferative disorders; splenectomy; and lung diseases or disorders such as emphysema, chronic bronchitis, breathing disorders associated with sleep apnea, chronic obstructive pulmonary disease, interstitial lung disease, alveolar hypoventilation disorders, chronic exposure to high altitude, and developmental abnormalities in the lung. Thus, an individual at risk for developing PH or an individual suspected of having PH includes an individual suffering from a heart disease, an individual that is prone to developing blood clots, an individual suffering from congestive heart failure, an individual having birth defects in the heart, or an individual suffering from chronic pulmonary thromboembolism, AIDS, cirrhosis of the liver, lupus, pulmonary fibrosis, collagen vascular disease, portal hypertension, a thyroid disorder; glycogen storage disease, Gaucher's disease, hereditary hemorrhagic telangiectasia, a hemoglobinopathy, a chronic myeloproliferative disorder; splenectomy; or a lung diseases or disorder such as emphysema, chronic bronchitis, breathing disorders associated with sleep apnea, chronic obstructive pulmonary disease, interstitial lung disease, alveolar hypoventilation disorders, chronic exposure to high altitude, and developmental abnormalities in the lung. In some instances, PH is a genetic disorder. Thus, an individual at risk for developing PH or an individual suspected of having PH also includes an individual who has a family member that suffers from PH (e.g., grandparent, parent, sibling, uncle, aunt, child, etc.).

In some embodiments, the individual to be treated has pulmonary hypertension (PH). In some embodiments, the individual to be treated is suspected of having PH. In some embodiments, the individual to be treated is at risk for developing PH (e.g., the individual can be at risk, or can be suspected of being at risk, for developing PH). In some embodiments, the individual to be treated has RV dysfunction, is suspected of having RV dysfunction, or is at risk for developing RV dysfunction, e.g. the individual has PH or is at risk for developing PH.

Any individual having PH or at risk for developing PH may be treated by the subject methods. The terms “individual”, “recipient”, “subject”, “host”, and “patient”, are used interchangeably herein and refer to any mammalian subject for whom diagnosis, treatment (e.g., preventing, treating, etc.), or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

By “treating” and “treatment”, it is generally meant obtaining a desired pharmacologic and/or physiologic effect on PH and, where necessary, RV dysfunction. The effect can be prophylactic in terms of completely or partially preventing the disorder or symptom(s) thereof, and/or may be therapeutic in terms of a partial or complete stabilization or cure for the disorder and/or adverse effect attributable to the disorder or symptom(s) thereof. More broadly, the term “treatment” encompasses any treatment of a disease and/or related symptom(s) in a mammal, particularly a human, and includes: (a) preventing the disease and/or symptom(s) from occurring in a subject who may be predisposed to the disease and/or symptom(s) but has not yet been determined to have the disease and/or symptom(s) (e.g., an individual suspected of having the disease and/or symptoms(s)); (b) inhibiting progression (i.e., worsening) of the disease and/or symptom(s), i.e., arresting development of a disease and/or the associated symptom(s), i.e., stabilizing a disease and/or the associated symptoms; or (c) relieving the disease and the associated symptom(s), i.e., causing regression (i.e., reversal) of the disease and/or symptom(s). As described above, those in need of treatment can include those already inflicted (e.g., those with PH, those with RV dysfunction, etc.) as well as those in which prevention is desired. By a “therapeutic treatment” it is meant a treatment in which the individual is inflicted (e.g., has PH, has RV dysfunction) prior to administration of the treatment. By a “prophylactic treatment” it is meant a treatment in which the subject is not inflicted (e.g., does not have PH, does not have RV dysfunction, etc.) prior to administration of the treatment. In some embodiments, the individual has an increased likelihood of becoming inflicted (e.g., the individual has risk factors predisposing them to PH and/or RV dysfunction) or is suspected of being inflicted (e.g., but has not been diagnosed) prior to treatment. In some embodiments, the individual is suspected of having an increased likelihood of becoming inflicted.

### Regulatory Macrophages (Mregs)

In practicing the subject methods, a cell composition that is enriched for regulatory macrophages (Mregs) is administered to the individual in an amount effective to, for example, prevent, stabilize, or reverse PH, and/or RV dysfunction, e.g. as measured by one or more of the above-mentioned parameters or parameters known in the art.

By “regulatory macrophages” or “Mregs”, it is meant a type of leukocyte of the monocyte lineage. Mregs suitable for use in the subject methods can be readily identified by the expression—or in some instances, absence of expression—of one or more of the following marker proteins: CD14, CD86, HLA-DR, MHC-II, CD80, CD40, CD11b, CD11c, F4/80, CD16, CD64, TLR2, TLR4, CD163, and/or CD274 (see, e.g., the working examples herein and Hutchinson et al., J Immunol. 2011 Sep. 1; 187(5):2072-8 and Brem-Exner et al., J Immunol. 2008 Jan. 1; 180(1):335-49). In some embodiments, the subject Mregs comprise an expression profile that is CD11c+ and CD274+. In some embodiments, the subject Mregs comprise an expression profile that is CD14^{-/low} MHC-II⁺ CD40^{lo} CD86^{lo} CD11b⁺ F4/80⁺ CD274⁺ and CD11c⁺. In some embodiments, the subject Mregs comprise an expression profile that is CD14^{-/low}, HLA-DR⁺, CD80^{-/low}, CD86⁺, CD16⁻, CD64⁺, TLR2⁻, TLR4⁻, and CD163^{-i/low}. In vitro, regulatory macrophages are readily identifiable by a flattened, spindled morphology, e.g. as described in Waldo S W, et al. Heterogeneity of human macrophages in culture and in atherosclerotic plaques. Am J Pathol, 2008; 172(4):1112-1126, the full disclosure of which is incorporated herein by reference.

By an enriched cell composition of Mregs, it is meant that at least about 70%, about 75%, or about 80% of the cells of the cell composition are Mregs, more usually at least 85% or 90% of the population are Mregs. In some instances, the enriched composition will be a substantially pure population of Mregs, whereby “substantially pure” it is meant at least 95% or more of the composition will be of the selected phenotype, e.g. 95%, 98%, and up to 100% of the population.

It will be understood by those of skill in the art that expression levels reflect detectable amounts of the marker (e.g., protein or nucleic acid) on and/or in the cell. A cell that is negative for staining (e.g., the level of binding of a marker specific reagent is not detectably different from a matched control) may still express minor amounts of the marker. And while it is commonplace in the art to refer to cells as “high”, “+”, “positive”, “low”, “−”, or “negative” for a particular marker, actual expression levels are quantitative traits. For example, number of detected molecules can vary by several logs, yet still be characterized as “positive”.

When a protein marker is used, the staining intensity (e.g., of a marker-specific antibody) can be monitored by any method suitable for assaying protein expression, e.g., flow cytometry, Western blotting, mass spectrometry, and enzyme-linked immunosorbent assay (ELISA).

As one example, in flow cytometry, lasers detect the quantitative levels of fluorochrome (which is proportional to the amount of cell marker bound by specific reagents, e.g. antibodies). Flow cytometry, or FACS, can also be used to separate cell populations based on the intensity of binding to a specific reagent (or combination of reagents), as well as other parameters such as cell size and light scatter. Although the absolute level of staining may differ with a particular fluorochrome and reagent preparation, the data can be normalized to a control. As such, a population of cells can be enriched for Mregs by using flow cytometry to sort and collect those cells (e.g., only those cells) with an Mreg profile.

In order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining brighter than that of an isotype matched control, but is not as intense as the most brightly staining cells normally found in the population. An alternative control may utilize a substrate having a defined density of marker on its surface, for example a fabricated bead or cell line, which provides the positive control for intensity.

Cell compositions that are enriched for Mregs that find use in the subject methods include compositions comprising Mregs that have been acutely isolated from an individual, e.g., the individual undergoing treatment, or a donor individual. Alternative, the subject cell compositions may be prepared in vitro by isolating a population of leukocytes comprising monocytes from an individual, e.g., the individual undergoing treatment, or a donor individual, and culturing the population in vitro to produce a cell composition that is enriched for Mregs. By monocytes it is meant a type of leukocyte (white blood cell) that is part of the innate immune system of vertebrates. Monocytes have bean-shaped nuclei and constitute 2-10% of all leukocytes in the human body. Monocytes are part of the myeloid lineage, and can act as precursor cells that replenish macrophages and/or dendritic cells under normal states. In response to inflammation, monocytes can move quickly to sites of infection and divide/differentiate into macrophages and/or dendritic cells (e.g., to elicit an immune response). Monocytes can be identified by their large kidney shaped or notched nucleus, as well as by the expression of certain cell surface markers including, for example, CD14. In some instances, the subject cell compositions that are enriched for Mregs are prepared from a heterogeneous population of leukocytes comprising monocytes, that is, a population in which about 60% or less, e.g. 50% or less, 40% or less, 30% or less, 20% or less, or 10% or less of the cells are monocytes. In other instances, the subject cell compositions may be prepared from an enriched population of monocytes, e.g. a population of leukocytes in which about 60% or more, e.g. 70% or more, 75% or more, 80% or more, 85% or more, 90% or more, 95% or more, 98% or more, in some instances substantially all of the cells are monocytes. Cell populations comprising monocytes may be obtained by any convenient method. For example, the monocytes may be obtained from blood (e.g., heparinized blood), bone marrow, and/or spleen tissues. The monocytes may be obtained from peripheral blood mononuclear cells (PBMCs) by Ficoll density gradient separation (e.g., leukapheresis followed by Ficoll density gradient separation). In some instances, affinity reagents, e.g. antibodies specific for monocyte cell-surface markers, e.g. CD14, may be employed.

In some instances, the donor of the Mregs or of the population of leukocytes comprising monocytes from which the subject cell compositions will be prepared is the same as the individual (the “recipient”) receiving the subject treatment (i.e., the individual suffering from or at risk of suffering from PH). In other words, the Mregs (or monocytes to be induced to differentiation into Mregs) are drawn from an individual as a blood draw, and the Mregs (or Mregs induced from monocytes) are transferred back (restored) into the same individual. In such an instance, the Mregs or monocytes to be induced to differentiation into Mregs are autologous to the recipient.

In other instances, the donor of the Mregs or of the population of leukocytes comprising monocytes from which the subject cell compositions will be prepared is different from the individual (the “recipient”) receiving the subject treatment (i.e., the individual suffering from or at risk of suffering from PH). In other words, the Mregs or monocytes to be induced to differentiation into Mregs are allogeneic to the recipient. In such instances, the Mregs or monocytes to be induced to differentiation into Mregs are selected based upon the blood type of the donor and the blood type of the recipient. By blood type, it is meant the presence or absence of A and B antigens and Rh antigen on the donor and recipient's red blood cells. For example, as is well understood in the art, an individual may have neither A or B antigens on his red blood cells (and hence will have antibodies specific for both A and B antigens in his plasma), in which case the individual is “type O”. The individual may have A antigen and not B antigen on his red blood cells (and hence will have antibodies specific for B antigen but not A antigen in his plasma), in which case the individual is “type A.” The individual may have B antigen and not A antigen on his red blood cells (and hence antibodies specific for A antigen but not B antigen in his plasma), in which case the individual is “type B.” The individual may have both A and B antigens on his red blood cells (and hence no antibodies for either A or B antigen in his plasma), in which case the individual is “type AB.” As well known in the art, safe transfusion of donor blood to a recipient can occur if the donor is type O and the recipient is any type; if the donor is type A and the recipient is type A or type AB; if the donor is type B and the recipient is type B or type AB; or if the donor is type AB and the recipient is type AB. Additionally, as is known in the art, the Rh antigen may or may not be present, i.e., the individual is Rh-positive or Rh-negative, respectively. As is well known in the art, safe transfusion of donor leukocytes to a recipient can occur if the donor is type Rh⁺ or Rh⁺ and the recipient is type Rh⁺; or if the donor is type Rh⁻ and the recipient is type Rh⁻.

Any convenient method for producing Mregs in vitro, e.g. as known in the art or as described in the working examples herein, may be used to produce the subject cell compositions. See, for example, Brem-Exner et al., J Immunol. 2008 Jan. 1; 180(1):335-49: “Macrophages driven to a novel state of activation have anti-inflammatory properties in mice.” and Hutchinson et al., “Human Regulatory Macrophages”; in Methods in Molecular Biology, vol. 677, Maria Cristina Cuturi and Ignacio Anegon (eds), the disclosures of which are incorporated herein by reference.

For example, monocytes may be induced to differentiate into Mregs by culturing in medium containing macrophage colony-stimulating factor (M-CSF) and interferon gamma (IFN-gamma). In other words, Mregs can be derived from monocytes when monocytes are contacted with M-CSF and IFN-gamma. In some cases, the monocytes are contacted for a period of time with M-CSF prior to being contacted with IFN-gamma (i.e., monocytes are cultured in M-CSF prior to culture in IFN-gamma). In some embodiments, the subject methods include preparing a cell population that is enriched for Mregs. In such embodiments, any convenient method of producing a cell population enriched for Mregs can be employed.

For example, monocytes (e.g., plastic adherent monocytes) may be cultured in the presence of M-CSF, e.g., in a basal medium such as RPMI 1640, for a period of time in a range of from 3 days to 10 days, e.g, 3 days to 9 days, 3 days to 8 days, 3 days to 7 days, 3 days to 6 days, 4 days to 10 days, 4 days to 9 days, 4 days to 8 days, 4 days to 7 days, 4 days to 6 days, 4.5 days to 5.5 days, 5 days to 10 days, 5 days to 9 days, 5 days to 8 days, 5 days to 7 days, 5 days to 6 days, 6 days to 10 days, 6 days to 9 days, 6 days to 8 days, 6 days to 7 days, 7 days to 10 days, 7 days to 9 days, 7 days to 8 days, 3 days, 4 days, 5 days, 6 days, 7 days, 8 days, 9 days, or 10 days. Any convenient M-CSF can be used. Suitable examples of M-CSF include, but are not limited to: recombinant M-CSF; recombinant human M-CSF; recombinant mouse M-CSF; recombinant rat M-CSF; and the like. In some embodiments, monocytes are contacted with M-CSF at a concentration in a range of from 2 ng/ml to 8 ng/ml, e.g., 2.5 ng/ml to 7.5 ng/ml, 3 ng/ml to 7 ng/ml, 3.5 ng/ml to 6.5 ng/ml, 4 ng/ml to 6 ng/ml, 4.5 ng/ml to 5.5 ng/ml, or 5 ng/ml. In some cases, the M-CSF can be carried on serum albumin, e.g., human serum albumin, e.g., 0.1% human serum albumin.

In some cases, monocytes are cultured in the presence of M-CSF in a basal medium, e.g., an RPMI 1640-based medium. In some cases, monocytes are cultured in the presence of M-CSF in a basal medium (e.g., an RPMI 1640-based medium) that does not include phenol red. In some cases, the medium further includes one or more of serum (e.g., 10% human AB serum); L-glutamine (e.g., 2 mM L-glutamine); penicillin (e.g., 100 U/ml); and streptomycin (e.g., 10 mg/ml).

The cells can be plated at any convenient density. In some cases, the cells are cultured at a density in a range of from 1×10⁵ to 1×10⁹ monocytes per 175 cm² (e.g., 5×10⁵ to 5×10⁸ monocytes per 175 cm²; 1×10⁶ to 2×10⁸ monocytes per 175 cm²; 5×10⁶ to 1×10⁸ monocytes per 175 cm²; 8×10⁶ to 6×10⁷ monocytes per 175 cm²; 1×10⁷ to 6×10⁷ monocytes per 175 cm²; 2×10⁷ to 5×10⁷ monocytes per 175 cm²; or 2.5×10⁷ to 4×10⁷ monocytes per 175 cm²).

In some cases, cultures are gently washed to select for adherent cells and fresh medium can then be added to the adherent cell layer. For example, in some cases, after monocytes have been contacted with M-CSF for a period of time in a range of from 12 hours to 36 hours (e.g., 18 hours to 30 hours, 20 hours to 28 hours, 22 hours to 26 hours, or 24 hours) cultures can be gently washed to select for adherent cells and fresh medium can then be added to the adherent cell layer. Such washes and selection can be repeated at any convenient interval thereafter (e.g., every 12 hours, every 24 hours, every 36 hours, every 48 hours, and the like).

In some embodiments, interferon gamma (IFN-gamma) (e.g., mouse recombinant IFN-gamma, recombinant human IFN-gamma, etc.) can be added to the culture at a concentration in a range of from 15 ng/ml to 35 ng/ml (e.g., 17.5 ng/ml to 32.5 ng/ml, 20 ng/ml to 30 ng/ml, 22.5 ng/ml to 27.5 ng/ml, 24 ng/ml to 26 ng/ml, or 25 ng/ml). The cultured cells can be contacted with IFN-gamma for a period of time in a range of from 12 hours to 36 hours (e.g., 14 hours to 32 hours, 14 hours to 30 hours, 14 hours to 28 hours, 14 hours to 24 hours, 14 hours to 22 hours, 15 hours to 32 hours, 15 hours to 30 hours, 15 hours to 28 hours, 15 hours to 24 hours, 15 hours to 21 hours, 16 hours to 30 hours, 16 hours to 28 hours, 16 hours to 24 hours, or 16 hours to 20 hours).

In some cases, the monocytes are cultured in the presence of IFN-gamma following a period time in which the monocytes were cultured in the presence of M-CSF. In some cases, monocytes are cultured in the presence of IFN-gamma and in the absence of M-CSF. For example, in some cases, monocytes are cultured in the presence of M-CSF for a period of time as described above, and then cultured in the absence of M-CSF but in the presence IFN-gamma for a period of time as described above. In some cases, monocytes are cultured in the presence of M-CSF for a period of time as described above, and then cultured in the presence of both IFN-gamma and M-CSF for a period of time as described above for IFN-gamma. For example, in some cases, monocytes are cultured in the presence of M-CSF for a period of time in a range of from 3 days to 10 days (e.g, 3 days to 9 days, 3 days to 8 days, 3 days to 7 days, 3 days to 6 days, 4 days to 10 days, 4 days to 9 days, 4 days to 8 days, 4 days to 7 days, 4 days to 6 days, 4.5 days to 5.5 days, 5 days to 10 days, 5 days to 9 days, 5 days to 8 days, 5 days to 7 days, 5 days to 6 days, 6 days to 10 days, 6 days to 9 days, 6 days to 8 days, 6 days to 7 days, 7 days to 10 days, 7 days to 9 days, 7 days to 8 days, 3 days, 4 days, 5 days, 6 days, 7 days, 8 days, 9 days, or 10 days) and then cultured in the presence of both M-CSF and IFN-gamma for a period of time as described above for a period of time in a range of from 12 hours to 36 hours (e.g., 14 hours to 32 hours, 14 hours to 30 hours, 14 hours to 28 hours, 14 hours to 24 hours, 14 hours to 22 hours, 15 hours to 32 hours, 15 hours to 30 hours, 15 hours to 28 hours, 15 hours to 24 hours, 15 hours to 21 hours, 16 hours to 30 hours, 16 hours to 28 hours, 16 hours to 24 hours, or 16 hours to 20 hours).

In some cases, adherent cells are harvested (e.g., with a cell scraper, using trypsin-EDTA treatment, and the like) and washed (e.g. in phosphate buffered saline (PBS); physiological salin solution, e.g., containing 5% human albumin for infusion; and the like) before use. Mregs for administration to human individuals can be prepared under strict Good Manufacturing Practice (GMP) conditions.

Other standard cell culture components that are suitable for inclusion in the culturing process include, but are not limited to, a vitamin; an amino acid (e.g., an essential amino acid); a pH buffering agent; a salt; an antimicrobial agent (e.g., an antibacterial agent, and antimycotic agent, etc.); serum; an energy source (e.g., a sugar); a nucleoside; a lipid; trace metals; a cytokine, a growth factor, a stimulatory factor, and the like. Any convenient cell culture media can be used. Various cell types grow better in particular media preparations (in some cases, particular media formulations have been optimized to culture specific types of cells (e.g., neurons, cardiomyocytes, hepatocytes, monocytes, Mregs, etc.). Accordingly, any convenient cell culture media can be used and may be tailored to the particular cell type being cultured.

The major ions and their concentrations in cell culture media are generally present in standard, commercially available, liquid culture media (e.g., basal liquid culture media). Most standard types of media (e.g., DMEM, DMEM/F12. BME, RPM 1640, and the like) use relatively narrow and fixed ranges for the concentrations of bulk ions in general and the monovalent cations Na⁺ and K⁺ in particular. This is in line with the fact that the ionic balance of the bulk ions in general and the monovalent cations Na⁺ and K⁺ in particular is a rather universal property of almost all mammalian cells. Any convenient media that can be used to culture cells in vitro is suitable for use with the subject compositions and methods.

In accordance with the typical concentration of sodium ions inside and outside a generic mammalian cell (Alberts et al., Molecular Biology of the Cell (1994)) mostly sodium concentrations of about 145 mM are chosen together with potassium ion concentrations of around 5 mM. For most media types this results in a ratio between sodium and potassium ions that ranges between about 20-30 (see U.S. Pat. No. 5,135,866; and US Patent Publication No. 2013/0122543, both of which are hereby incorporated by reference in their entirety).

In some embodiments, a subject cell culture medium (e.g., a basal culture medium) includes animal serum (e.g., fetal bovine serum (FBS); fetal calf serum (FCS), bovine serum, chicken serum, newborn calf serum, rabbit serum, goat serum, normal goat serum (NGS); horse serum; lamb serum, porcine serum, human serum (e.g., human AB serum, AB-human serum, and the like). A wide range of serum concentrations can be used. A cell culture composition of the present disclosure can have a concentration of serum in a range of from 1% to 50% (e.g., from 2% to 40%, from 2% to 30%, from 2% to 25%, from 2% to 20%, from 2% to 15%, from 2% to 10%, from 2% to 7%, from 2% to 5%, from 3% to 12%, from 5% to 15%, from 8% to 12%, from 9% to 11%, from 8% to 20%, 5%, 6%, 7%, 8%, 9%, 10%, 11%, 12%, 13%, 14%, or 15%). In some embodiments, a subject cell culture medium is serum free. Serum comprises growth factors and in many cases, is it unknown exactly which growth factors, or exactly how many growth factors are present in any given serum. In some cases, at least one of the growth factors present in a serum is known.

The population of Mregs so prepared will be an enriched population of Mregs. In other words, at least about 70%, about 75%, or about 80% of the cells of the population be of the selected phenotype, more usually at least 85% or 90% of the population be of the selected phenotype. In some instances, the enriched population will be a substantially pure population of Mregs, whereby “substantially pure” it is meant at least 95% or more of the population be of the selected phenotype, e.g. 95%, 98%, and up to 100% of the population. In some instances, it may be advantageous to mechanically enrich for (i.e., purify) the Mregs. For example, if an acutely isolated population of Mregs is to be employed, it may be necessary to mechanically enrich the population for Mregs to provide for an enriched population of Mregs. As another example, when culturally enriched Mregs are to be employed (e.g., Mregs prepared by culturing monocytes ex vivo), it may be desirable to further enrich the population of Mregs, for example, to arrive at a substantially enriched population of Mregs. By “mechanical enriching” it is meant a mechanical separation of cells of interest (e.g., Mregs) from a cell population, for example by positive selection of the cells of interest or by negative selection (depletion) of the cells not of interest. Examples of mechanical enrichment strategies include, but are not limited to: cell sorting using flow cytometry (e.g., fluorescence activated cell sorting (FACS)), cell sorting using magnetic bead sorting (e.g., magnetic beads conjugated to antibodies and/or ligands that bind to Mreg markers), immunopanning (e.g., using a solid support conjugated to antibodies and/or ligands that bind to Mreg markers), and the like. In particular aspects, subject Mregs may be selected or enriched by using a screenable or selectable reporter expression cassette comprising an Mreg-specific transcriptional regulatory element operably linked to a reporter gene. Because some Mregs can exist in a sample prior to culture, a subject method for producing Mregs (as described above) is considered for the purposes of this disclosure to be an example of enriching a cell population for Mregs. Thus “enriching” refers to a step in which the fraction (i.e., percentage) of Mregs present in the final cell population is greater than the fraction of Mregs present in the starting cell population.

In some cases, multiple types of enriching can be used. In some cases, enriching can happen in more than one step. For example, in some cases, monocytes are cultured for a period of time in the presence of M-CSF (in M-CSF without IFN-gamma, M-CSF with IFN-gamma, etc.). In some cases, this is followed by culture for a period of time in the presence of INF-gamma (e.g., in the presence or absence of M-CSF). A step of mechanical enrichment can be performed at any point in the process. For example, cells can be mechanically sorted (e.g., the cell population can be enriched for Mregs, e.g. using a affinity reagents specific fro Mregs, e.g. CD14-specific antibodies or a CD14+ column) prior to culture in the presence of M-CSF or at point after culture has commenced. In some cases, after monocytes are enriched for Mregs and the enriched population is cultured in order to proliferate (e.g., in the presence or absence of M-CSF). In some cases, after monocytes are cultured in the presence of M-CSF and cultured in the presence of IFN-gamma, the resulting cell population is then enriched for Mregs by mechanical enrichment (e.g., cells of the cell population are subjected to flow cytometry) to further enrich the cell population for Mregs. In some cases, the cell population is enriched for Mregs after the monocytes are cultured in the presence of M-CSF, but prior to culture in the presence of IFN-gamma. Enrichment using antibodies (e.g., magnetic cell sorting, FACS, and the like) specific for cell surface markers of Mregs have the advantage of not requiring genetic modification of the cells to be enriched. Magnetic cell sorting and FACS have the ability to analyze multiple surface markers simultaneously, and they can be used to sort Mregs based on the expression levels of cell surface markers.

Once produced, the presence and/or percent of Mregs in the population can be readily verified by, for example, detecting the expression of the one or more proteins of the Mreg expression profile, e.g. CD14, CD86, HLA-DR, MHC-II, CD80, CD40, CD11b, CD11c, F4/80, CD16, CD64, TLR2, TLR4, CD163, and/or CD274. In some embodiments, the subject methods include the step of verifying the presence of Mregs in a cell population, e.g., after culturing as descried above, by detecting the expression of one or more of CD14, CD86, HLA-DR, MHC-II, CD80, CD40, CD11b, CD11c, F4/80, CD16, CD64, TLR2, TLR4, CD163, and/or CD274, wherein CD14^{-/low}, MHC-II⁺, CD40^{low}, CD86^{low}, CD11b^{low}, F4/80⁺, CD274⁺, and CD11c^{+/high} are indicative of Mregs. In some embodiments, the subject methods include the step of verifying the presence of Mregs in a cell population, e.g., after culturing as descried above, by detecting the expression of one or more of CD14, CD86, HLA-DR, MHC-II, CD80, CD40, CD11b, CD11c, F4/80, CD16, CD64, TLR2, TLR4, CD163, and/or CD274, wherein CD14^{-/low}, HLA-DR⁺, CD80^{-/low}, CD86⁺, CD16⁻, CD64⁺, TLR2⁻, TLR4⁻, and CD163^{-i/low} are indicative of Mregs.

Additionally or alternatively, verifying can rely on cellular phenotypes, e.g., gene or protein expression, drug metabolism profile, responsiveness to particular drugs, etc., that are characteristic of Mregs. Marker expression (e.g., as determined by measuring protein and/or RNA levels) may be examined before, during, and/or after the production of Mregs by the subject methods. The expressed set of markers may be compared against other subsets of cells (e.g., untreated precursor monocyte cells). In some cases, cells of a subject population are assayed in order to measure the percent of cells in the population that are Mregs. In some cases, a cell population is enriched for Mregs, which increases the percent of cells of a cell population that are Mregs. In some cases, enriching occurs simultaneously verifying the presence of the Mregs (e.g., when using flow cytometry to enrich a cell population for Mregs).

In some embodiments, verifying includes contacting cells of a cell population with specific binding agents (e.g., an antibodies, nucleic acid probes, etc.) that are specific for Mreg markers (e.g., protein, mRNA etc.) and determining the percentage of cells of the cell population that are Mregs (e.g., the percentage of cells that have an Mreg profile, as discussed above). Suitable markers are listed above. In some cases, 10% or more of the cells of a cell population are determined to be Mregs (e.g., 10.5% or more, 11% or more, 12.5% or more, 15% or more, 17.5% or more, 20% or more, 22.5% or more, 25% or more, 27.5% or more, 30% or more, 32.5% or more, 35% or more, 37% or more, 40% or more, 45% or more, 50% or more, 55% or more, 60% or more, 65% or more, 70% or more, 75% or more, 80% or more, 85% or more, 90% or more, 95% or more, 98% or more, 99% or more, or 100%). In some embodiments, the percent of cells of the cell population that are determined to be Mregs is in a range of from 10% to 100%, e.g., from 20% to 100%, from 30% to 100%, from 40% to 100%, from 50% to 100%, from 60% to 100%, from 70% to 100%, from 75% to 100%, from 80% to 100%, from 85% to 100%, from 90% to 100%, or from 95% to 100%. Verification of the presence of Mregs can be performed at any point in the process of producing Mregs. For example, the percent of Mregs can be determined on any day during culture in the presence of M-CSF, any day during culture in the presence of IFN-gamma, after culturing cells in the presence of IFN-gamma, and/or after a step of enrichment (e.g., mechanical enrichment).

In some instances, the level of expression of the one or more Mreg markers is determined by detecting protein. Any method for detecting Mreg markers at the protein level, e.g. as known in the art or as described above, e.g., flow cytometry, Western blotting, mass spectrometry, and enzyme-linked immunosorbent assay (ELISA), may be employed.

In some instances, the level of expression of an Mreg marker is determined by detecting nucleic acid (e.g., mRNA). Any suitable qualitative or quantitative methods known in the art for detecting specific mRNAs can be used. mRNA can be detected by, for example, hybridization to a microarray, next-generation sequencing, in situ hybridization, by reverse transcriptase-polymerase chain reaction (rtPCR), or in Northern blots containing poly A mRNA. One of skill in the art can readily use these methods to determine differences in the size or amount of mRNA transcripts between two samples.

Any suitable method for detecting and comparing mRNA expression levels in a sample can be used in connection with the methods of the invention. For example, the mRNA from a sample can be sequenced via next-generation sequencing methods known in the art such as nanopore sequencing (e.g. as described in Soni et al Clin Chem 53: 1996-2001 2007, or as described by Oxford Nanopore Technologies), Illumina's reversible terminator method, Roche's pyrosequencing method (454), Life Technologies' sequencing by ligation (the SOLID platform) or Life Technologies' Ion Torrent platform. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009; 513:19-39) and Morozova (Genomics. 2008 92:255-64), which are incorporated by reference for the general descriptions of the methods and the particular steps of the methods, including all starting products, reagents, and final products for each of the steps.

### Therapeutic Methods

In practicing the subject methods, a cell composition that is enriched for Mregs, e.g. prepared by the methods described herein, is administered to the individual in an amount effective to prevent or treat Pulmonary Hypertension and the right ventricular dysfunction that may ensue. By “effective amount”, “therapeutically effective dose” or “therapeutic dose” it is meant the amount sufficient to effect desired clinical results (i.e., achieve therapeutic efficacy). For purposes of this disclosure, a therapeutically effective dose of the subject cell composition (i.e., the cell composition that is enriched for Mregs) is an amount that is sufficient, when administered to (e.g., transplanted into) the individual, to palliate, ameliorate, stabilize, reverse, prevent, slow or delay development and/or progression of Pulmonary Hypertension, and if RV dysfunction has developed in the individual, to palliate, ameliorate, stabilize, reverse, prevent, slow or delay development and/or progression of RV dysfunction in about 1-60 days, e.g. 2 days, 5 days, 7 days, 10 days, 15 days, 20 days, 25 days, 30 days, 35 days, 40 days, 50 days, or 60 days. An amount may be readily determined as being an effective amount by assaying for the symptoms of PH, e.g., measuring mean pulmonary arterial pressure, measuring capillary wedge pressure, assessing for shortness of breath during routine activity, for tiredness/fatigue, for chest pain or pressure, for a rapid heartbeat, for swelling of the ankles, legs, and abdomen, for dizziness, for decreased appetite, and/or by assaying RV function, e.g. by assessing remodeling of the pulmonary artery, ventricular structure, ventricular remodeling, RV-PA coupling, etc. as described herein and as known in the art.

In some embodiments, a therapeutically effective dose of the subject cell compositions is about 1×10³ or more cells, for example, 5×10³ or more, 1×10⁴ cells, 5×10⁴ or more, 1×10⁵ or more, 5×10⁵ or more, 1×10⁶ or more, 5×10⁶ or more, 1×10⁷ cells, 5×10⁷ or more, 1×10⁸ or more, 5×10⁸ or more, 1×10⁹ or more, 5×10⁹ or more cells, and usually not more than 1×10¹⁰ cells. In some embodiments, a therapeutically effective dose of cells (e.g., Mregs) is in a range of from about 1×10³ cells to about 1×10¹⁰ cells, e.g, from about 5×10³ cells to about 1×10¹⁰ cells, from about 1×10⁴ cells to about 1×10¹⁰ cells, from about 5×10⁴ cells to about 1×10¹⁰ cells, from about 1×10⁵ cells to about 1×10¹⁰ cells, from about 5×10⁵ cells to about 1×10¹⁰ cells, from about 1×10⁶ cells to about 1×10¹⁰ cells, from about 5×10⁶ cells to about 1×10¹⁰ cells, from about 1×10⁷ cells to about 1×10¹⁰ cells, from about 5×10⁷ cells to about 1×10¹⁰ cells, from about 1×10⁸ cells to about 1×10¹⁰ cells, from about 5×10⁸ cells to about 1×10¹⁰, from about 5×10³ cells to about 1×10⁹ cells, from about 1×10⁴ cells to about 1×10⁹ cells, from about 5×10⁴ cells to about 1×10⁹ cells, from about 1×10⁵ cells to about 1×10⁹ cells, from about 5×10⁵ cells to about 1×10⁹ cells, from about 1×10⁶ cells to about 1×10⁹ cells, from about 5×10⁶ cells to about 1×10⁹ cells, from about 1×10⁷ cells to about 1×10⁹ cells, from about 5×10⁷ cells to about 1×10⁹ cells, from about 1×10⁸ cells to about 1×10⁹ cells, from about 5×10⁸ cells to about 1×10⁹, from about 5×10³ cells to about 1×10⁸ cells, from about 1×10⁴ cells to about 1×10⁸ cells, from about 5×10⁴ cells to about 1×10⁸ cells, from about 1×10⁵ cells to about 1×10⁸ cells, from about 5×10⁵ cells to about 1×10⁸ cells, from about 1×10⁸ cells to about 1×10⁸ cells, from about 5×10⁶ cells to about 1×10⁸ cells, from about 1×10⁷ cells to about 1×10⁸ cells, from about 5×10⁷ cells to about 1×10⁸ cells, from about 5×10³ cells to about 5×10⁷ cells, from about 1×10⁴ cells to about 5×10⁷ cells, from about 5×10⁴ cells to about 5×10⁷ cells, from about 1×10⁵ cells to about 5×10⁷ cells, from about 5×10⁵ cells to about 5×10⁷ cells, from about 1×10⁸ cells to about 5×10⁷ cells, from about 5×10⁶ cells to about 5×10⁷ cells, or from about 1×10⁷ cells to about 5×10⁷ cells. The ordinarily skilled artisan will appreciate that a therapeutically effective dose can be administered in one or more administrations.

The number of administrations of the subject cell composition to achieve treatment in a subject may vary. For example, in some instances, only one administration of the subject cell composition may be required. In other instances, such treatment may elicit improvement for a limited period of time and require an on-going series of repeated treatments. In some situations, multiple administrations of cells may be required before an effect is observed. As will be readily understood by one of ordinary skill in the art, the exact protocols depend upon the disease or condition, the stage of the disease and parameters of the individual being treated.

The subject cell composition may be introduced by any convenient method (e.g., injection, catheter, or the like). The cells may be administered to the subject (i.e., introduced into the individual) via any of the following routes: parenteral, subcutaneous, intravenous, intracranial, intraspinal, or intraocular. Examples of methods for cell delivery include, e.g., by bolus injection, e.g. by a syringe, e.g. into a joint or organ; e.g., by continuous infusion, e.g. by cannulation, e.g. with convection (see e.g. US Application No. 20070254842, incorporated here by reference); or by implanting a device and/or matrix upon which the cells have been reversably affixed (see e.g. US Application Nos. 20080081064 and 20090196903, incorporated herein by reference).

The subject cell composition may be administered systemically or locally (e.g., administered into the heart, near the heart, into the pulmonary tissue, etc.). When administered systemically, the subject cell composition will typically be administered intravascularly, e.g. intravenously or intraarterially. The subject cell composition may be mixed with intravascular solutions as known in the art, e.g. 5% dextrose in water, an isotonic electrolyte solution such as isotonic saline (0.9%), etc. The subject cell composition may be administered using any convenient access device, e.g. needle for intravenous injection, compressor gun, peripheral cannula, central IV line, etc., e.g. implantable port, tunneled line, central venous lines, peripherally inserted central catheters and the like. When administered locally, the subject cell composition may be administered by any convenient method that provides for the localized placement of cells in heart or pulmonary tissue, for example, injection or transplantation into an airway, e.g. endotracheal or endobronchial injection, or direct parenchymal injection via endoscopic or open delivery tools.

In some instances, the cells are administered in a suspension. In other instances, the cells are co-administered with, e.g. in association with, or concurrently with, a suitable substrate or matrix, e.g. to support their survival, growth, organization, etc. In some embodiments, the matrix is a scaffold (e.g., an organ scaffold, e.g., bone marrow scaffold). In some cases, the matrix is a biodegradable matrix (e.g., a biodegradable scaffold). In some cases, the cells can exit the matrix and migrate to another location within the individual (e.g., enter the blood stream, enter the bone marrow, etc.). A suitable support matrix can be derivatized with functional groups such as recombinant proteins, positively charged tertiary quaternary or primary amines, gelatin, collagen, other extracellular matrix (ECM) proteins and peptides (e.g. RGD peptide).

Particular examples of degradable scaffolds include an albumin scaffold, a fibrin scaffold or a combination thereof. A degradable albumin scaffold can be formed by mixing equal portions of two solutions to form a cross-linked gel. The first solution can contain human serum albumin and the second solution can contain the cross-linking agent, illustratively including modified poly(ethylene glycol), PEG, glutaraldehyde or transglutaminase at a concentration of approximately 0.10 g/ml as detailed in U.S. Pat. No. 6,656,496, which is hereby incorporated by reference in its entirety. Still other albumin-based scaffolds are detailed in US 20050069589, which is hereby incorporated by reference in its entirety. When the two solutions are mixed in a ratio approaching stoichiometry, a gel forms. The gel can be made porous by adding unmodified PEG particles. This albumin scaffold degrades in vivo by both enzymatic and hydrolytic degradation. For more information about scaffolds (e.g., bioadhesive scaffolds), refer to U.S. patent application number 20090232784, which is hereby incorporated by reference in its entirety.

In some cases, the support matrix is a hydrogel matrix. Hydrogel polymers may include one or more of a monomer, including, but not limited to: lactic acid, glycolic acid, acrylic acid, 1-hydroxyethyl methacrylate (HEMA), ethyl methacrylate (EMA), propylene glycol methacrylate (PEMA), acrylamide (AAM), N-vinylpyrrolidone, methyl methacrylate (MMA), glycidyl methacrylate (GDMA), glycol methacrylate (GMA), ethylene glycol, fumaric acid, and the like. Common cross linking agents include tetraethylene glycol dimethacrylate (TEGDMA) and N,N′-methylenebisacrylamide. The hydrogel can be homopolymeric, or can comprise co-polymers of two or more of the aforementioned polymers. Suitable hydrogel polymers also include, but are not limited to, poly(N-isopropylacrylamide) (pNIPAAm); poly(N-isopropylacrylamide-co-acrylic acid); hyaluronic acid or hyaluronate; crosslinked hyaluronic acid or hyaluronate; pHEMA; or copolymers of p(NIPAAm)-based sIPNs and other hydrogel sIPNs (semi-interpenetrating networks). In certain embodiments, the hydrogel polymer is a hyaluronic acid (HyA) polymer, which is a natural glycosaminoglycan (GAG) biopolymer with a variety of favorable biological properties. In some embodiments, the hydrogel polymer is an acrylated hyaluronic acid (HyA) polymer. Suitable hydrogels and hydrogel compositions are also described in U.S. applications 20040001892, 20130184235, 20130183349, 20130276669, 20130267455, 20130244943, 20130136697, 20130129835, 20130129800, 20130045242, 20130012913, and 20130004546; all of which are hereby incorporated by reference in their entirety.

The cells of the disclosure (e.g., Mregs) may be administered in any physiologically acceptable excipient (e.g., William's E medium). The subject enriched population of Mregs can be supplied in the form of a pharmaceutical composition, e.g., comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

The subject enriched population of Mregs may be genetically altered in order to introduce genes useful in the differentiated hepatocytes, e.g. repair of a genetic defect in an individual, selectable marker, etc. Cells may also be genetically modified to enhance survival, control proliferation, and the like. Cells may be genetically altered by transfection or transduction with a suitable vector, homologous recombination, or other appropriate technique, so that they express a gene of interest. In some embodiments, a selectable marker is introduced, to provide for greater purity of the desired differentiating cell. In some cases Mregs are labeled, e.g., for tracking (e.g., labeled with luciferase, labeled with a fluorescent protein such as GFP, labeled with a radioactive isotope such as Indium-111, and the like).

### Compositions, Systems, Reagents and Kits

Aspects of the disclosure include cell compositions for preventing and treating pulmonary hypertension (PH) and right ventricular (RV) dysfunction. In some embodiments, the subject cell composition includes a composition of cells enriched for regulatory macrophages (Mregs). In some cases, of the composition of cells enriched for Mregs, 70% or more of the cells are Mregs, e.g., 75% or more, 80% or more, 85% or more, 90% or more, 95% or more, 98% or more, 99% or more, or 100% of the cells are Mregs. In some embodiments, a cell composition is a substantially pure population of Mregs, i.e., 95% or more of the population is Mregs, e.g., 98% or 100% of the population is Mregs. In some cases, the subject cell composition is in a form that is ready for administration into an individual, e.g., the cell composition is a pharmaceutical composition, as described above.

Also provided in some aspects of the invention are systems for preventing and treating pulmonary hypertension (PH) and right ventricular (RV) dysfunction. Such systems can include: (i) a population of leukocytes comprising monocytes from the individual (e.g., a population of PBMCs, an enriched population of monocytes, etc.); and (ii) a cell composition enriched for Mregs, where the Mregs are the in vitro progeny of the monocytes. For example, the enriched population of Mregs can be the cell composition for use in the subject methods. As another example, the population of monocytes can be any population of cells isolated during the process of producing the Mreg population. In some cases, the population of monocytes contains cells that have not yet been contacted with M-CSF. In some cases, the population of monocytes includes cells that have been contacted with M-CSF, but have not yet been contacted with IFN-gamma. In some cases, the population of monocytes includes cells that have been contacted with M-CSF and IFN-gamma, but have not been further enriched (e.g., mechanically enriched) for Mregs. In some cases, the population of monocytes contains cells that were mechanically sorted from a blood draw and then allowed to proliferate.

Also provided in some aspects of the invention are reagents, devices and kits thereof for practicing one or more of the above-described methods. The subject reagents, devices and kits thereof may vary greatly. Reagents and devices of interest include those mentioned above with respect to the methods of preparing an enriched population of Mregs for administration to a subject in need thereof. This would include, for example, reagents for isolating, purifying, and storing leukocytes from an individual, e.g. anti-coagulants, cryopreservatives, buffers, isotonic solutions, and the like; reagents for culturing Mregs ex vivo, e.g. M-CSF, IFN-gamma, suitable buffers, etc.; reagents for mechanically enriching for

Mregs, e.g., affinity reagents, magnetic beads, chromatography substrates, etc.; and reagents for confirming that the cell population to be administered is enriched for Mregs, e.g. antibodies, oligonucleotides, and the like specific for CD14, CD86, HLA-DR, MHC-II, CD80, CD40, CD11b, CD11c, F4/80, CD16, CD64, TLR2, TLR4, CD163, and/or CD274, e.g., a CD14-specific antibody or oligonucleotide, an HLA-DR-specific antibody or oligonucleotide, a CD86-specific antibody or oligonucleotide, a MHC-II-specific antibody or oligonucleotide, a CD80-specific antibody or oligonucleotide, a CD40-specific antibody or oligonucleotide, a CD11b-specific antibody or oligonucleotide, a CD11c-specific antibody or oligonucleotide, a F4/80-specific antibody or oligonucleotide, a CD16-specific antibody or oligonucleotide, a CD64-specific antibody or oligonucleotide, a TLR2-specific antibody or oligonucleotide, a TLR4-specific antibody or oligonucleotide, a CD163-specific antibody or oligonucleotide, and/or a CD274-specific antibody or oligonucleotide, affinity reagents of particular interest including those specific for CD11c and CD274.

Kits comprising combinations of these reagents and/or systems are also envisioned. Kits may also comprise blood collection bags, tubing, needles, centrifugation tubes, and the like. In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

As an imbalance between tissue-reparative process and inflammatory cascade may be detrimental for the failing myocardium, immunomodulation therapy using Mregs is shown in the following examples to improve RV function in the setting of persistent PH. The application of Mregs in heart failure can provide a new cell-based therapy for PH patients.

### Example 1

### RAT model of severe PH and RV failure

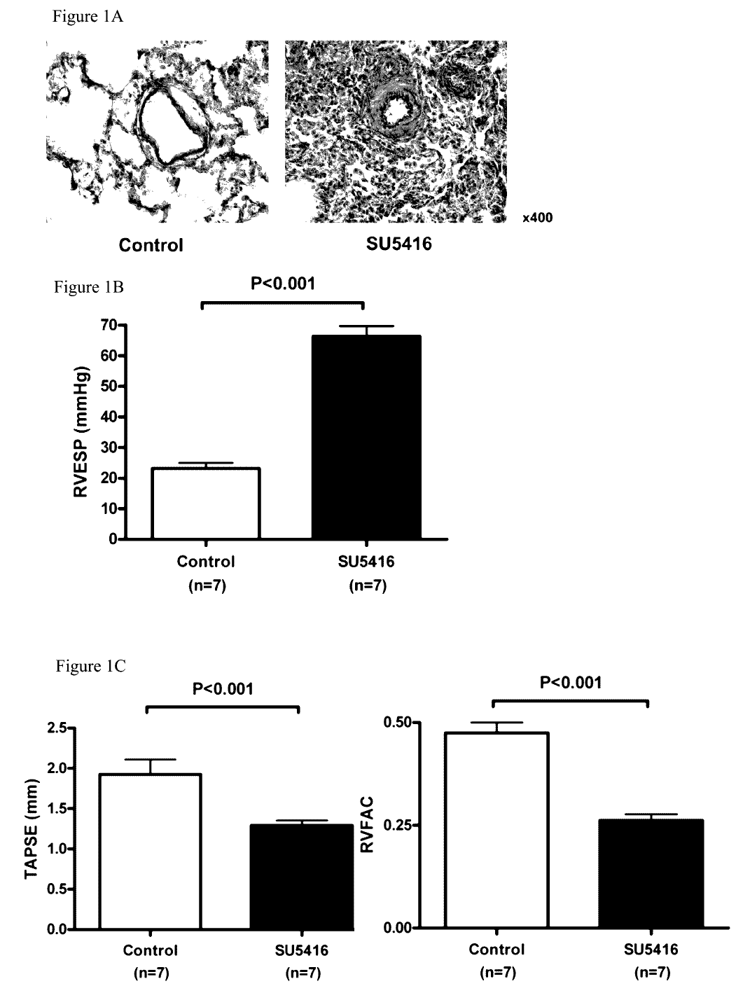

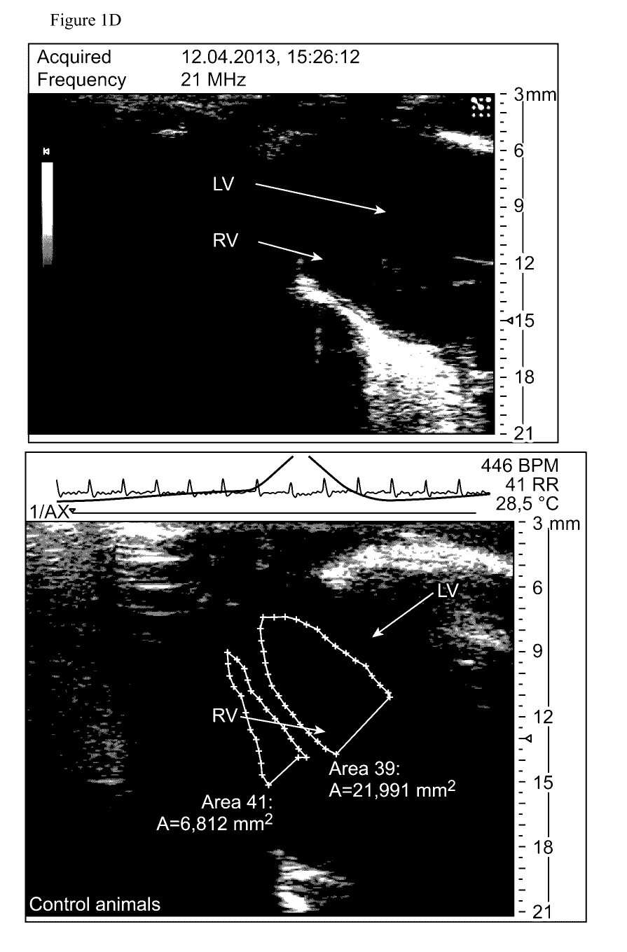

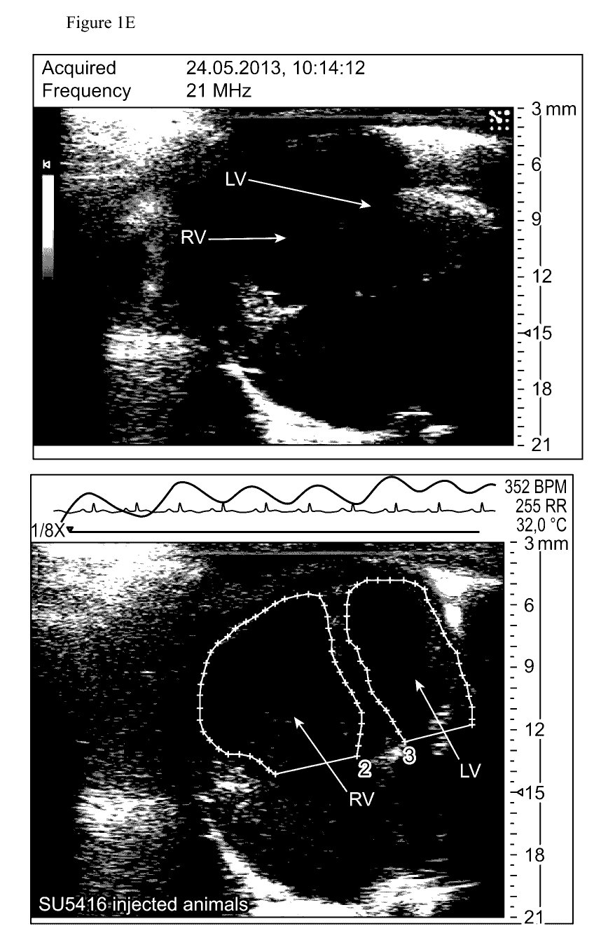

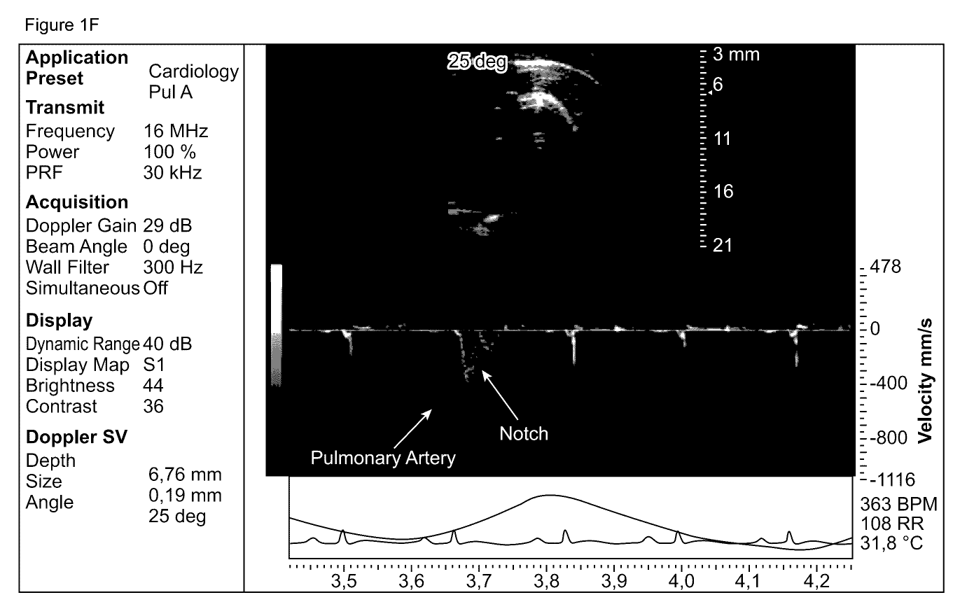

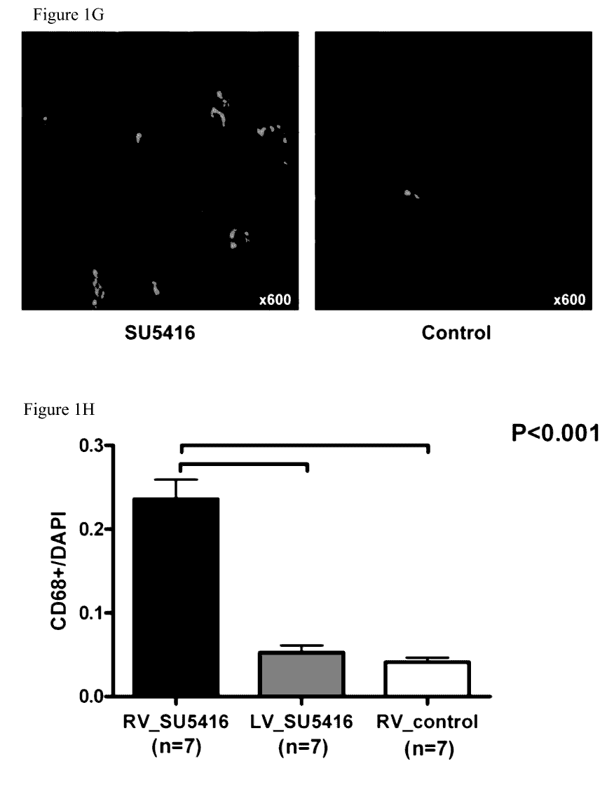

The immune insufficiency component of severe pulmonary hypertension (PH) can be modeled in athymic RNU rats (lacking T-lymphocytes). In comparison with other PH models, which require chronic hypoxia or a surgical pneumonectomy, T-cell deficiency renders the RNU athymic animals particularly sensitive to the development of severe PH under normoxic conditions. In these rats housed under normoxic conditions, treatment with SU5416 (a potent and selective inhibitor of the vascular endothelial growth factor (VEGF) receptor (Flk-1/KDR, a receptor tyrosine kinase)) resulted in severe PH, right ventricular (RV) dysfunction, and RV failure (RVF) within 28 days (FIG. 1). RVF was induced in the rats, which were on a 20 mg/kg copper diet, by a single subcutaneous injection (20 mg/kg) of SU5416 (semaxinib, SUGEN Inc). The rats developed significant RV remodeling, perivascular inflammation, and occlusive arteriolar lesions that are similar to lesion observed in severe clinical PH in humans. Similar results are achieved by a single subcutaneous injection (40 mg/kg) of SU5416 in the absence of copper in the diet.

As demonstrated in FIG. 1, SU5416 caused development of severe PH (severe RV dysfunction). This was characterized by the formation of occlusive neointimal and plexiform lesions in small peripheral pulmonary arteries (FIG. 1A). SU5416 injected animals developed significant RV dysfunction as quantified by RVESP (FIG. 1B). In addition, chronic pressure overload resulted in significant systolic dysfunction of the RV (TAPSE and RVFAC) at 4 weeks (FIG. 1C). Echocardiography shows the massive dilated right ventricle at 4 weeks after SU5416 injection (FIG. 1D, 1E). As demonstrated by Pulse Doppler of the pulmonary artery trunk, SU5416 injection causes increased pulmonary vascular resistance (FIG. 1F). Chronic pressure overload of the RV results in significant infiltration of CD68+ macrophages into the RV myocardium (4 weeks after SU5416 injection) (FIGS. 1G and 1H) Note, infiltration was not observed in the left ventricle wall, suggesting that the observations are not related to general SU5416 toxicity.

### Example 2

### Production and Characterization of Mregs

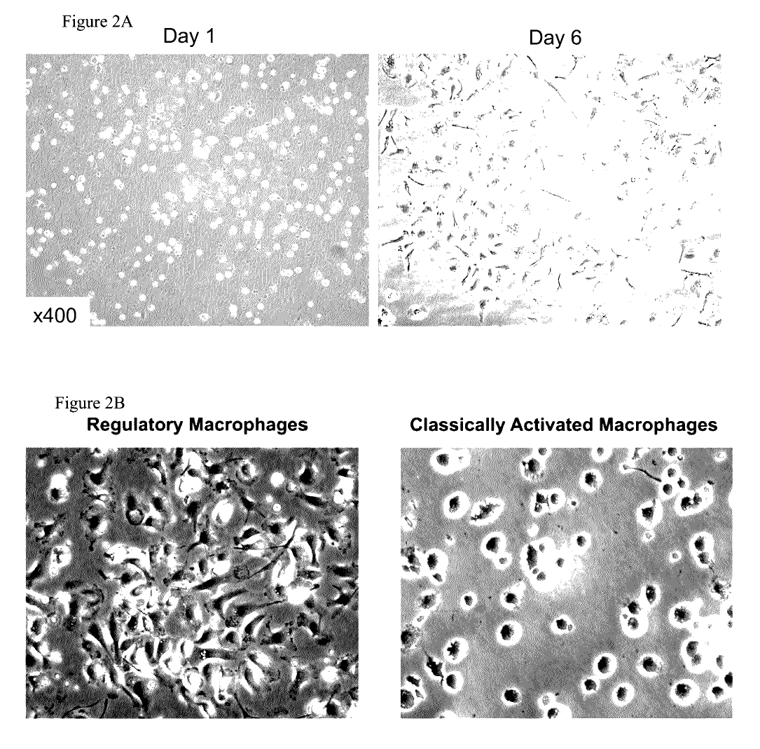

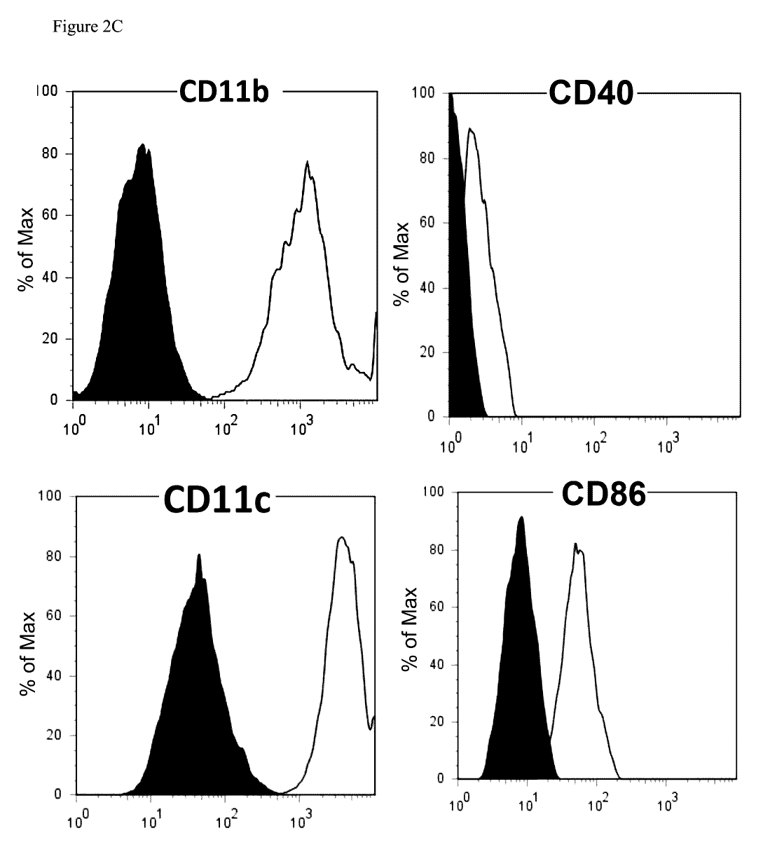

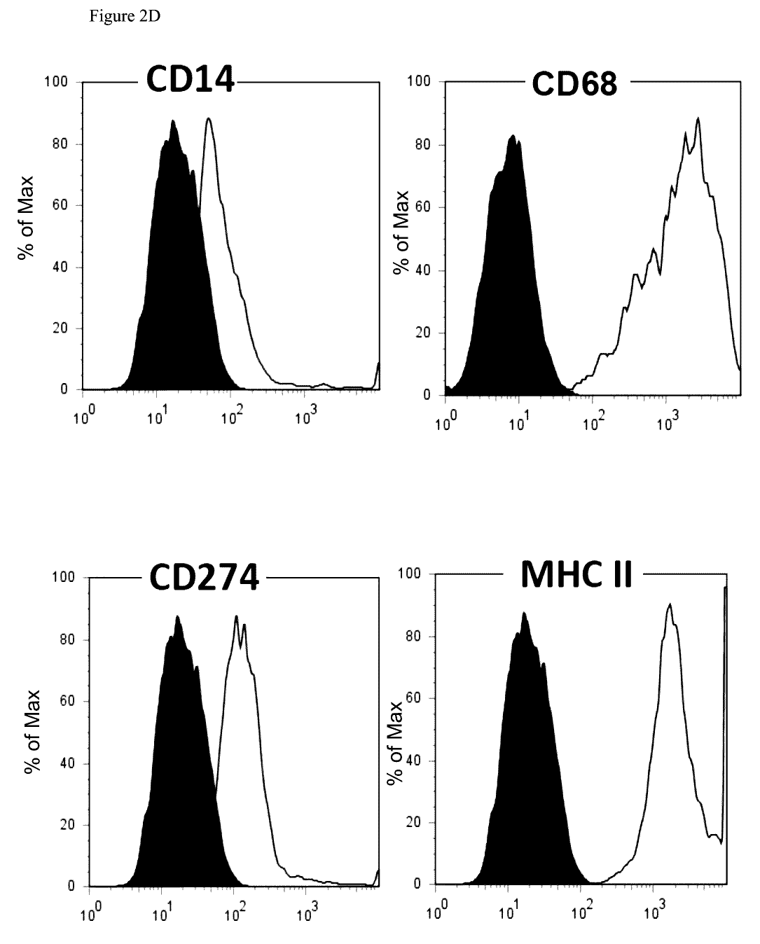

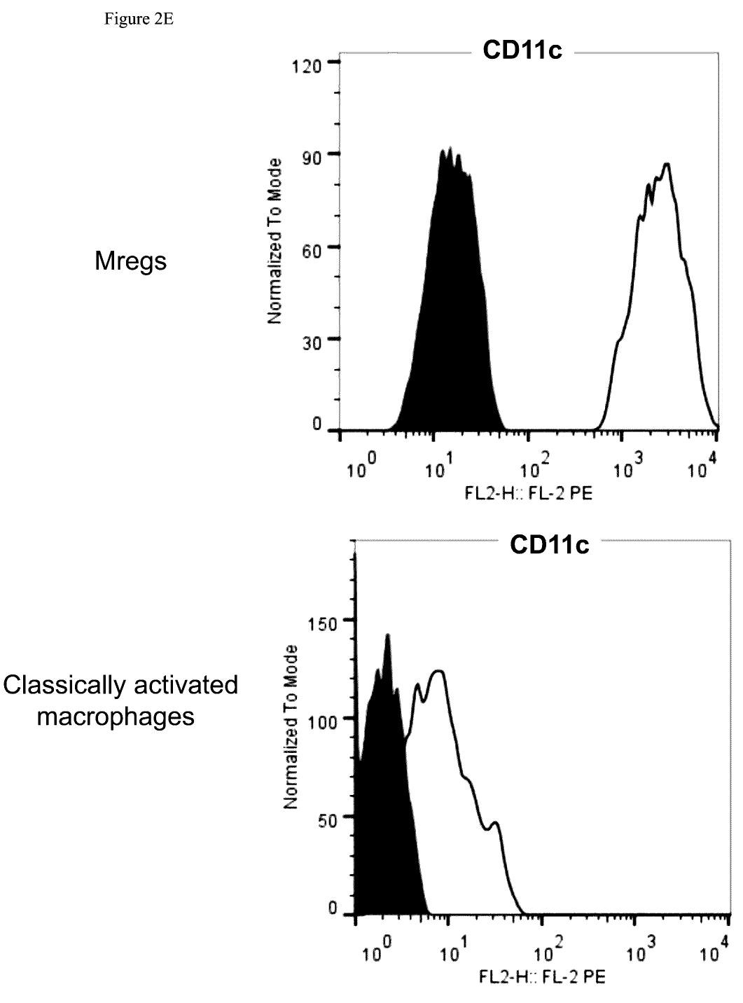

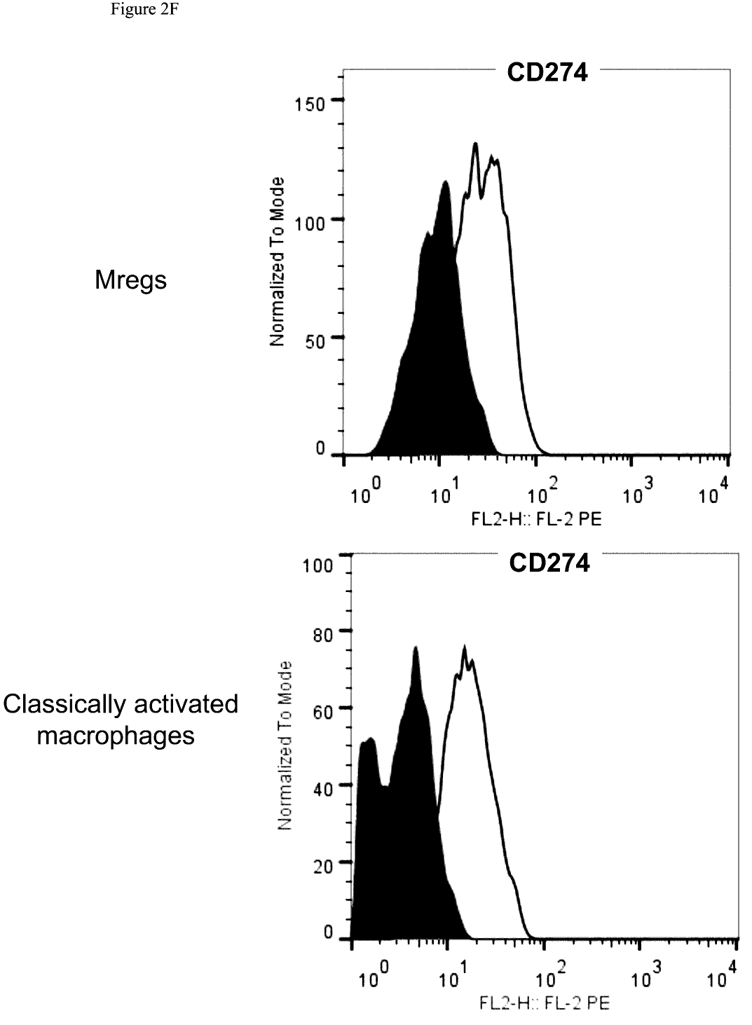

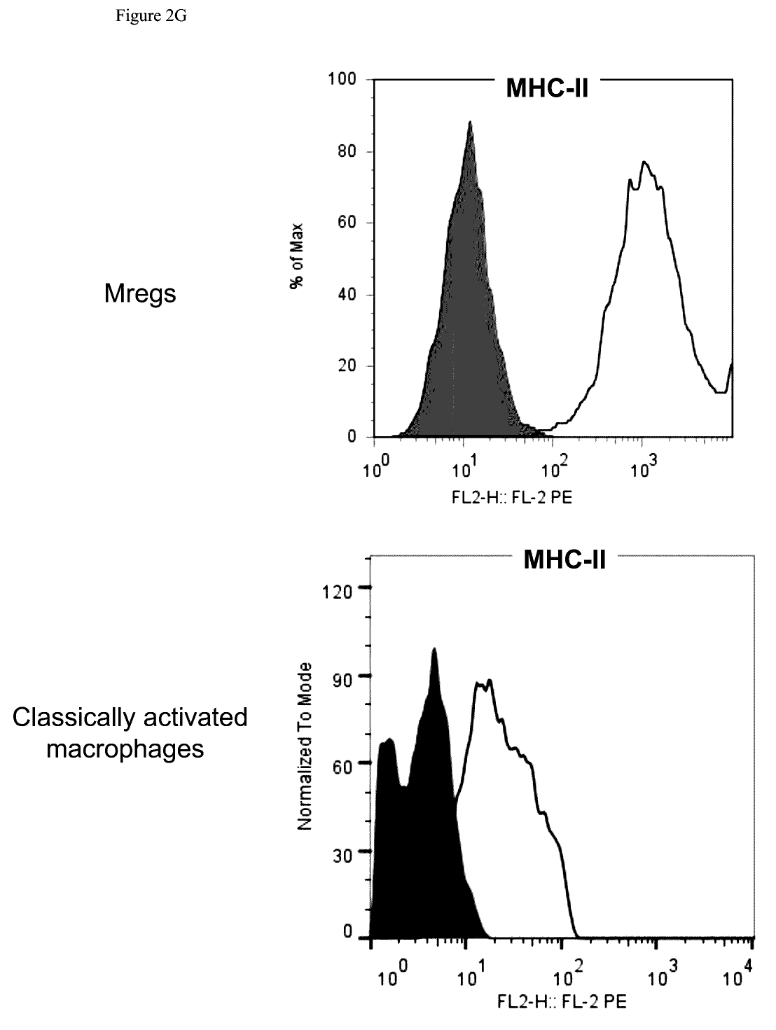

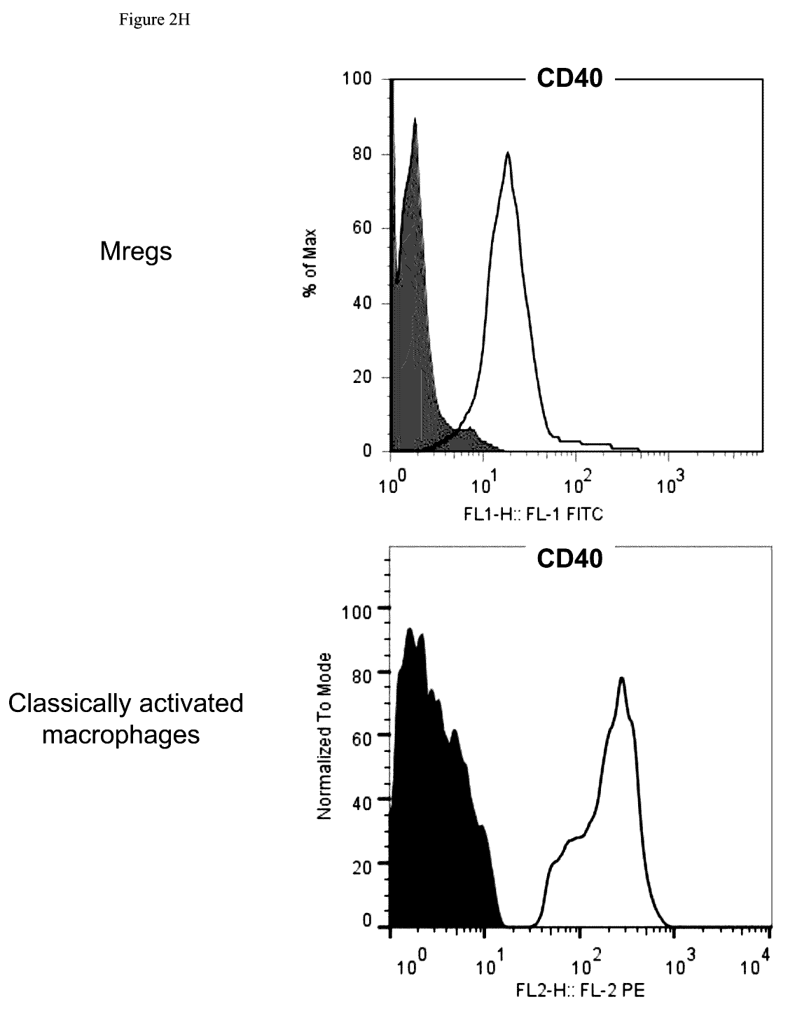

Mregs were generated in a protocol adapted from the published protocols of Brem-Exner et al. and Hutchinson et al. (Brem-Exner et al., J Immunol. 2008 Jan. 1; 180(1):335-49: “Macrophages driven to a novel state of activation have anti-inflammatory properties in mice.”; and Hutchinson et al., “Human Regulatory Macrophages”; in Methods in Molecular Biology, vol. 677, Maria Cristina Cuturi and Ignacio Anegon (eds.); which are hereby incorporated by reference in their entirety). Briefly, mononuclear cells were obtained from spleen and bone marrow of syngeneic donor rats by Ficoll density gradient separation. Cells were cultured for 5 days in RPMI 1640 medium containing 10% FCS and 5 ng/ml rat recombinant M-CSF. After 1 and 3 days of culture, cells were gently washed to select for plastic-adherent cells. On day 5, 25 ng/ml rat recombinant IFN-γ (interferon gamma) was added to the cultures for 16-20 hours. Adherent cells (Mregs; see FIG. 2A as an example) were harvested with a cell scraper. In some cases, Mregs were stably labeled with Fluc (luciferase) to track their survival, migration, and proliferation in vivo.

The Mreg populations generated were homogeneously CD14^{-i/low} MHC-II⁺ CD40^{lo} CD86^{lo} CD11b⁺ F4/80⁺ CD274⁺ and CD11e. This phenotype was stable from generation (Mreg production) on day 6 until approximately day 30 (Half-life varied between 10 and 30 days). Mregs exhibited a clear difference in morphology compared to classically activated macrophages (FIG. 2B). Although Mregs share markers of both resting and M1 macrophages, they can be readily distinguished from cells in these activation states. Mregs expressed higher levels of CD11c and MHC-II compared to both resting and M1 macrophages. Mregs expressed similar levels of CD11b, CD14, CD86, and F4/80 compared to resting macrophages, but relatively higher levels of CD40, CD274, and MHC class II. In contrast, Mregs expressed lower levels of CD40 and CD86 compared to M1 macrophages, which is consistent with the notion that these cells exist in a state of “partial maturation”. (see FIG. 2C-H).

### Example 3

### Prevention of RV Dysfunction and RV Failure with Mregs

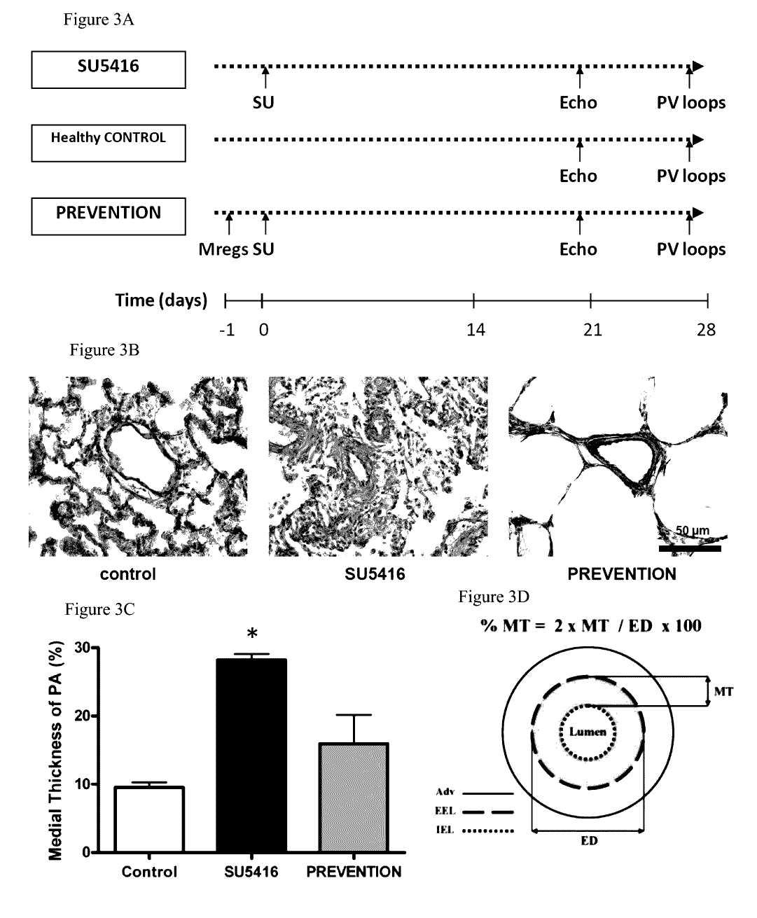

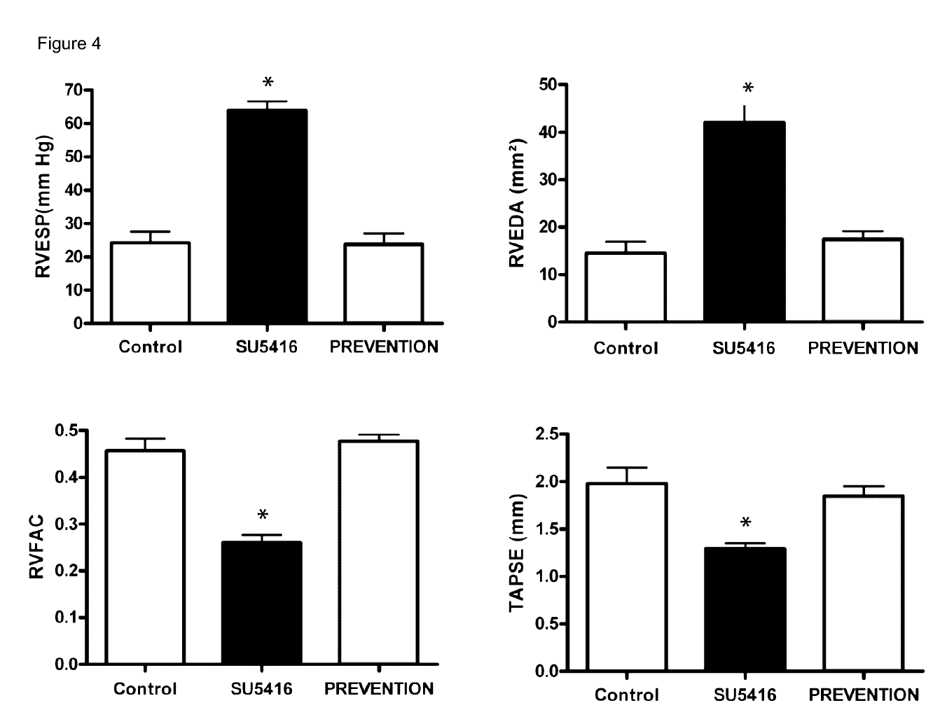

1×10⁶ Mregs were injected into the inferior vena cava of RNU rats (n=6) one day prior to injection of SU5416 (day “minus one” (−1)). One day later, PH and RVF were induced by SU5416 injection and animals were allowed to recover for 28 days (see FIG. 3A for experimental setup).

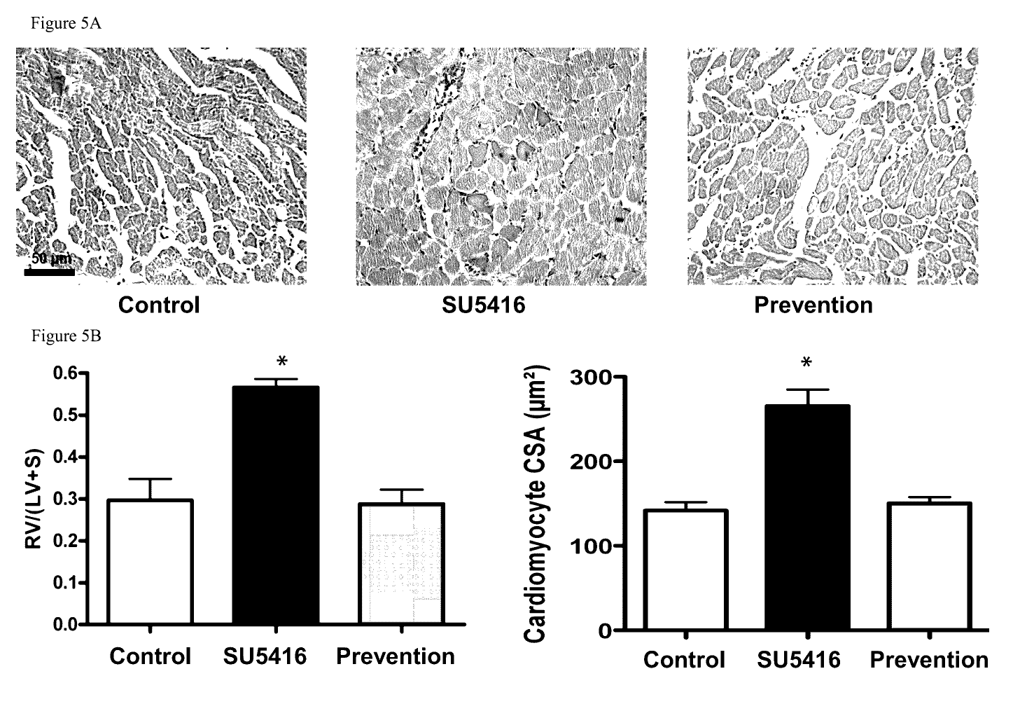

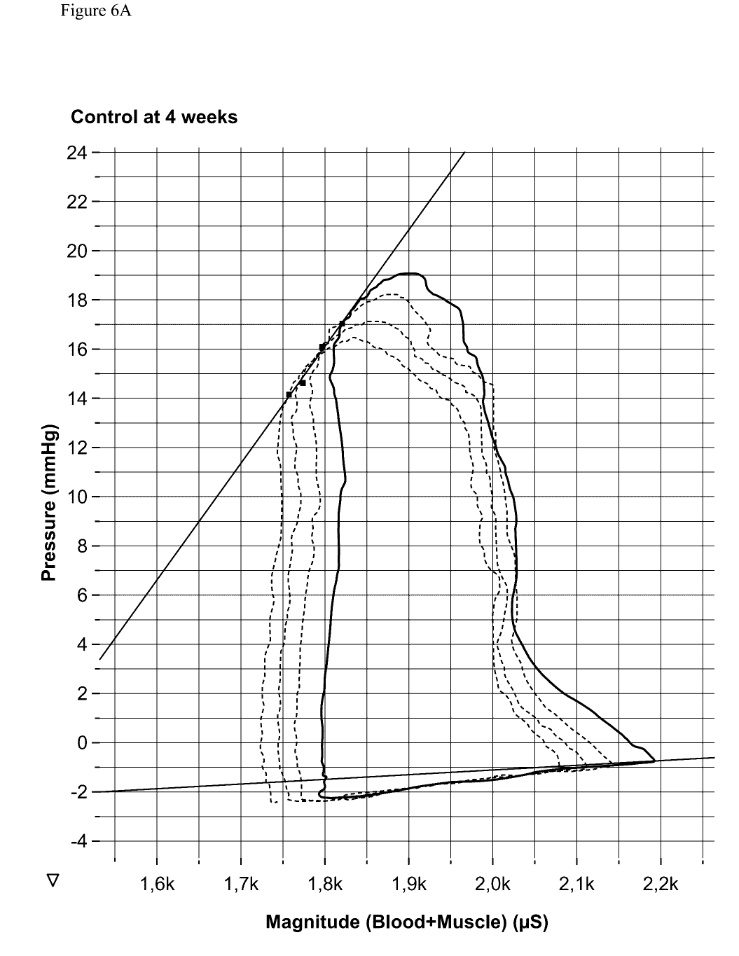

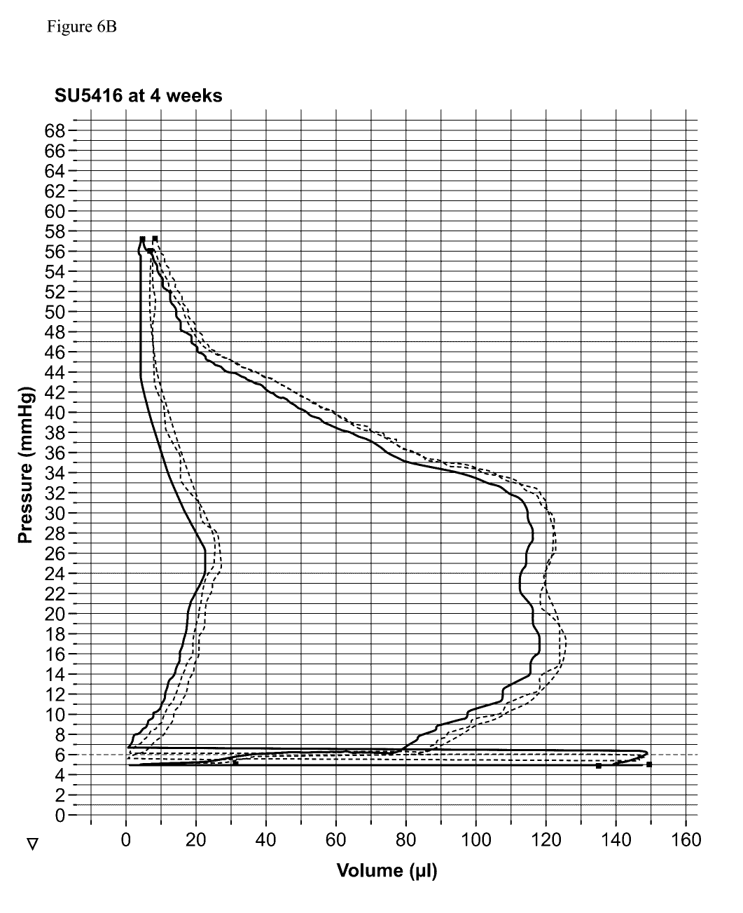

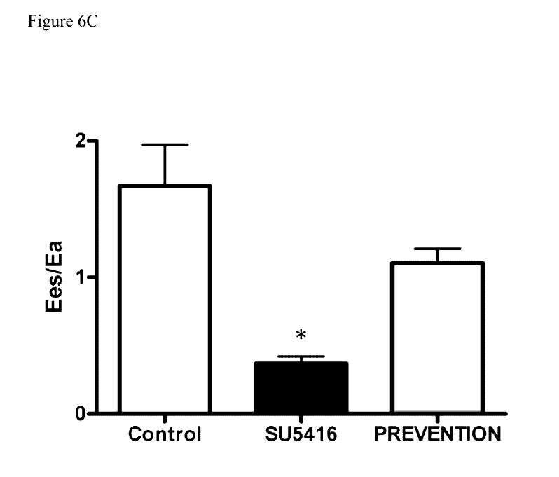

The data show that the injection of Mregs prevented right ventricular (RV) dysfunction and RV failure (RVF), as measured at day 28 by a number of different parameters related to: remodeling of the pulmonary artery (FIGS. 3B and 3C), ventricular structure and function (FIG. 4), ventricular remodeling (FIG. 5), and RV-PA coupling (FIG. 6). As such, injection of Mregs prevented the SU5416-induced: (i) medial thickening of the pulmonary aorta; (ii) increase in right ventricular end-systolic pressure (RVESP); (iii) decrease in right ventricular fractional area change (RVFAC); (iv) decrease in tricuspid annular plane systolic excursion (TAPSE); (v) increase in right ventricular end-diastolic area (RVEDA); (vi) increase in weight RV ratio (RV/(LV+S)); (vii) increase in cardiomyocyte area (e.g., cross-sectional area); and (viii) decrease in RV-PA coupling ratio (Ees/Ea).

### Example 4

### The Distribution of Injected Mregs as Tracked Over Time

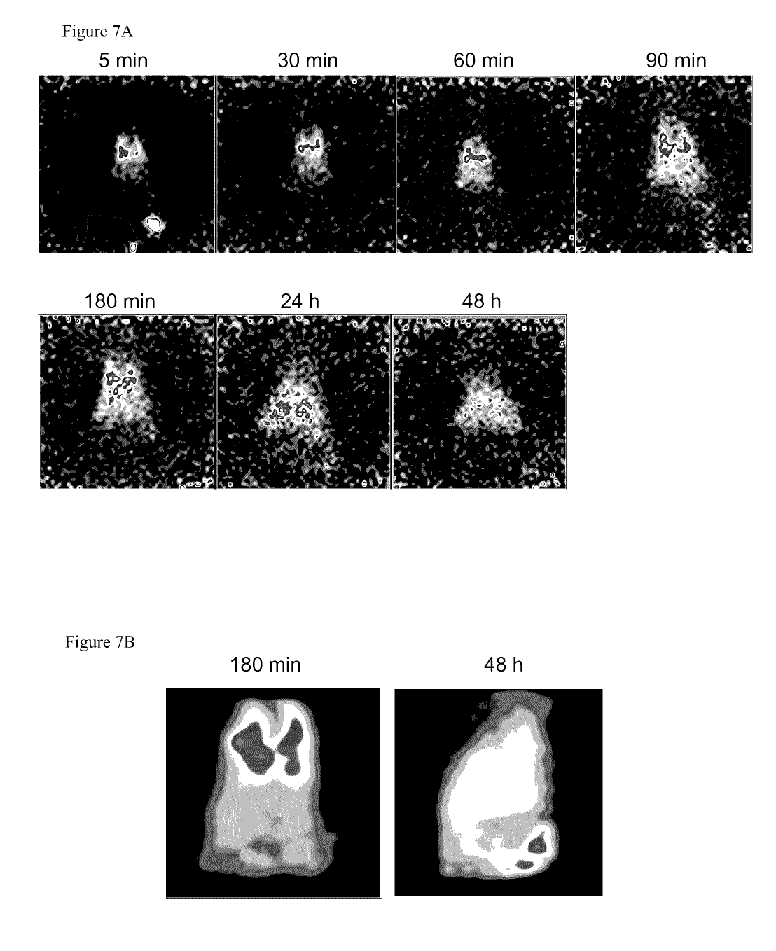

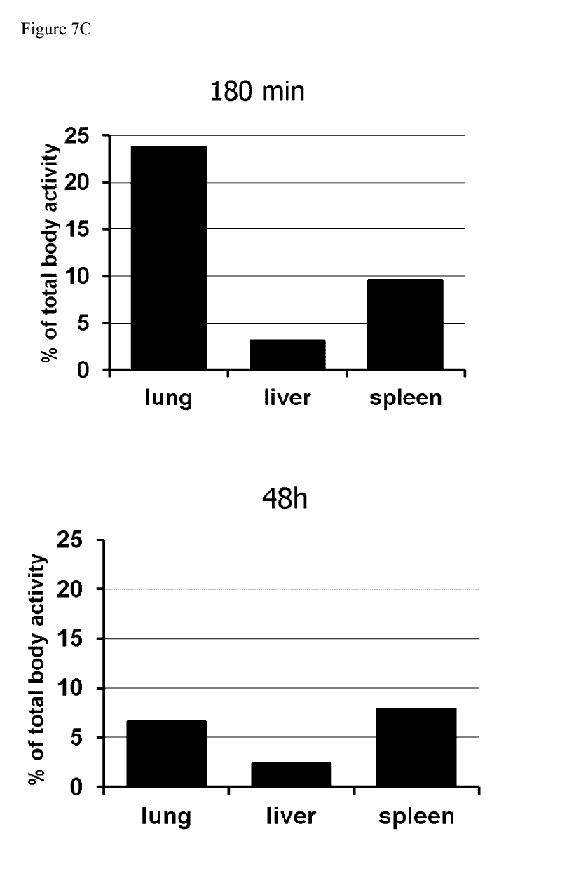

Mregs were injected into rats and were tracked over time. Indium-111 oxine (from GE Amersham; half-life of 2.8 days) was used to label Mregs prior to intravenous (i.v.) injection. Indium-111 remains trapped within the Mregs as long as the plasma membrane of the cell remains intact. Rats were imaged using single-photon emission computed tomography (SPECT) to detect the labeled Mregs. Injected Mregs were found in the lungs (even 5 minutes after injection). After 2 days, Mregs were found in lungs and also in the spleen (FIG. 7). The injected cells seem to remain in the animal for a short period of time (e.g., a few days), which is sufficient to inhibit the development of PH. It can be an advantage that injected cells do not survive for a very long period of time, given potential concerns regarding unwanted effects in the body (e.g. tumor induction or graft-versus-host disease, etc).

FIG. 7A depicts SPECT imaging (over time) demonstrating presence of the injected Mregs in the lungs. FIG. 7B shows the presence of Mregs in the lungs at 180 minutes (3 hours), and also spleen by 48 hrs. FIG. 7C shows quantification of the data (signal per organ per gram body weight of the animal).

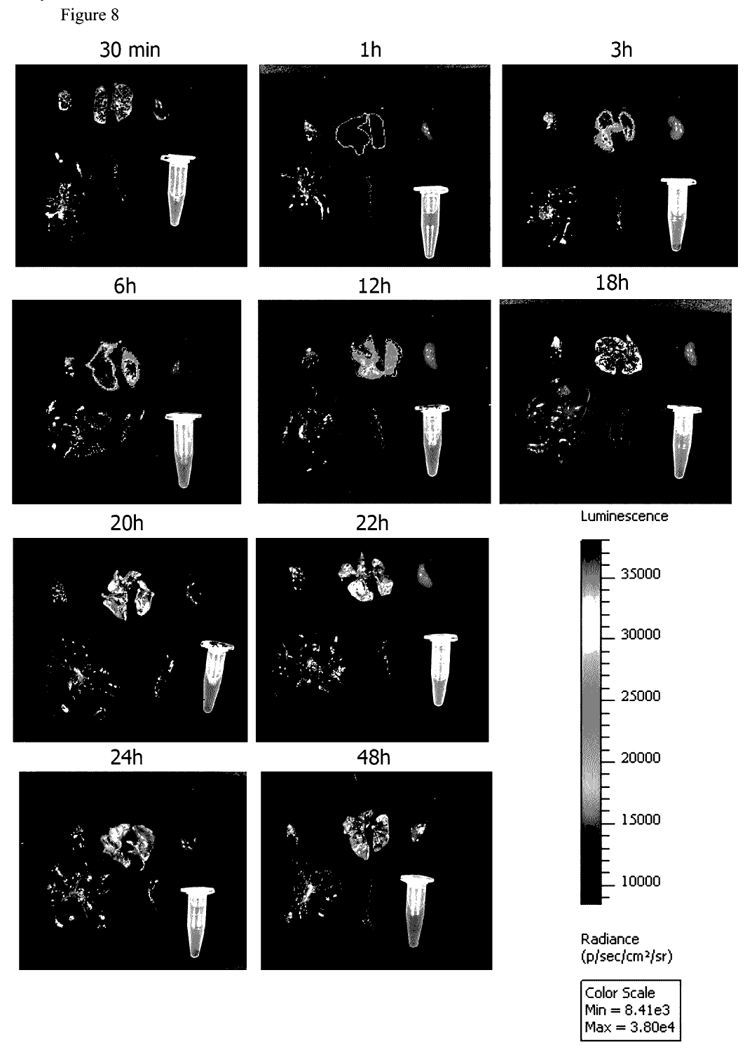

Mregs were also labeled with luciferase for bioluminescence (BLI) imaging and injected into Rats. Intravenous injected Mregs were detected in the lungs (FIG. 8), confirming the previous SPECT data of FIG. 7. After 20 h, no bioluminescence signal was detected. However, this result is likely due to a lack of sensitivity: BLI is not as sensitive a radioactive tracking. Also, the gamma related X-ray energy photons from Indium-111 have a far bigger punch per photon than visible light. For example, molecules labeled with an X-ray or PET isotope are about 2 to 3 orders of magnitude more sensitive than the BLI method.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of preventing or treating pulmonary hypertension (PH) in an individual, the method comprising:
administering to an individual having PH or at risk of developing PH a cell composition that is enriched for regulatory macrophages (Mregs) in an amount effective to prevent, stabilize, or reverse PH in the individual.

2. The method according to claim 1, further comprising, prior to administration, preparing the cell population that is enriched for regulatory macrophages (Mregs).

3. The method according to claim 2, wherein the producing comprises:
producing an enriched population of Mregs from a cell population comprising monocytes from the individual.

4. The method according to claim 3, wherein the producing comprises culturing the monocytes in vitro in the presence of macrophage colony-stimulating factor (M-CSF) and interferon gamma (IFN-gamma) to produce the enriched population of Mregs.

5. The method according to claim 4, wherein producing comprises mechanically enriching for Mregs.

6. The method according to claim 5, wherein the mechanically enriching comprises flow cytometry, magnetic bead sorting, or immunopanning.

7. The method according to claim 1, wherein the administering comprises systemic administration.

8. The method according to claim 1, wherein the individual has PH, wherein the method treats the PH.

9. The method according to claim 8, wherein the individual having PH has right ventricular (RV) dysfunction, wherein the method treats the RV dysfunction.

10. The method according to claim 9, wherein the method further comprises assaying for RV dysfunction before and after said administering, wherein the method stabilizes or reverses the RV dysfunction.

11. The method according to claim 8, wherein the individual is at risk for developing RV dysfunction, wherein the method prevents the development of the RV dysfunction.

12. The method according to claim 11, wherein the method further comprises assaying for RV dysfunction before and after said administering, wherein the method prevents the development of RV dysfunction.

13. The method according to claim 1, wherein the individual is at risk for developing PH, wherein the method prevents the development of PH.

14. A system for use in preventing or treating PH in an individual, the system comprising:
a population of cells comprising monocytes, and
an enriched population of regulatory macrophages (Mregs) that are the in vitro progeny of the monocytes.

15. The system according to claim 14, wherein the enriched population of Mregs is produced by a method comprising culturing the population of cells comprising monocytes in vitro in the presence of M-CSF and IFN-gamma.

16. A cell composition for use in preventing or treating PH in an individual, the composition comprising:
an enriched population of regulatory macrophages (Mregs) prepared by culturing a population of cells comprising monocytes in vitro in the presence of M-CSF and IFN-gamma.

17. The cell composition according to claim 16, wherein the monocytes are from the individual and the Mregs are autologous to the individual.

18. The composition according to claim 16, wherein the culturing comprises culturing in M-CSF followed by culturing in IFN-gamma.

19. A kit for use in determining if a cell population is an enriched population of Mregs, the kit comprising one or more antibodies selected from the group consisting of: a CD14-specific antibody, an HLA-DR-specific antibody, a CD86-specific antibody, a MHC-II-specific antibody, a CD40-specific antibody, a CD11b-specific antibody, a CD11c-specific antibody, a F4/80-specific antibody, a CD80-specific antibody, a CD16-specific antibody, a CD64-specific antibody, a TLR2-specific antibody, a TLR4-specific antibody, a CD163-specific antibody, a CD274-specific antibody, and a combination thereof.

20. The kit according to claim 19, wherein the kit comprises a CD14-specific antibody, a CD86-specific antibody, a MHC-II-specific antibody, a CD40-specific antibody, a CD11b-specific antibody, a CD11c-specific antibody, a F4/80-specific antibody, and a CD274-specific antibody.

21. The kit according to claim 19, wherein the kit comprises a CD11c-specific antibody and a CD274-specific antibody.
